(19) United States
(12) Patent Application Publication
(10) Pub. No.: US 2005/0228017 A1
(43) Pub. Date: Oct. 13, 2005

(54) Novel anticancer compounds

(75) Inventors: Sanjay R Menon, Plainsboro, NJ (US); Yingchun Lu, Kendall Park, NJ (US); Sukumar Sakamuri, Plainsboro, NJ (US); Quin-Zene Chen, Belle Mead, NJ (US); Vladimir Khazak, Brooklyn, NY (US)

Correspondence Address: PALMER & DODGE, LLP;PAULA CAMPBELL EVANS, 111 HUNTINGTON AVENUE, BOSTON, MA, 02199, US

(73) Assignee: Morphochem Aktiengesellschaft Fur Kombinatorische Chemie

(21) Appl. No.: 10/497,449

(22) PCT Filed: Oct. 31, 2002
(86) PCT No.: PCT/EP02/12222

§ 371 Date: Mar. 30, 2005

Related U.S. Application Data
(60) Provisional application No. 60/335,300, filed on Oct. 31, 2001.

(51) Int. Cl. A61K 31/455; A61K 31/445; C07D 213/46

(52) U.S. Cl. 514/317; 514/345; 514/355; 546/225; 546/315

(57) ABSTRACT

The present invention relates to compounds of formula (I): (I) their pharmacologically acceptable salts, or solvates and hydrates, and their prodrugs, respectively, and to pharmaceutical compositions containing the same as active ingredient. These novel compounds are especially useful in the treatment of cancer.

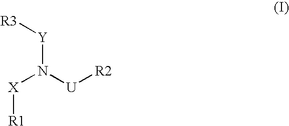

### FIELD OF THE INVENTION

The present invention relates to novel compounds, their pharmacologically acceptable salts, or solvates and hydrates, and their prodrugs, respectively, and to pharmaceutical compositions containing the same as active ingredient. These novel compounds are especially useful in the treatment of cancer.

### BACKGROUND OF THE INVENTION

Mitogen activated protein kinases or MAP kinases, a group of signal transduction proteins that are activated by cell surface receptors in response to the binding of growth factors, control many critical cellular functions, such as cell growth, cell differentiation and apoptosis (Z. Chen et al. “MAP Kinases” Chem. Rev. 2001, 101, 2449-2476). Raf, a kinase that phosphorylates MEKs (or MAP kinase kinases), and its upstream activator, Ras are two such protein interaction partners involved in the MAP kinase signalling cascade. Unregulated activation of Ras (derived from rat sarcoma), usually a result of a single point mutation in the Ras oncogene, is thought to be a significant contributor to cancer development, particularly colon and pancreatic cancers (A. Wittinghofer and H. Waldmann. “Ras-A Molecular Switch Involved in Tumor Formation” Angew. Chem. Int. Ed. 2000, 39, 4192-4214).

Ras farnesylation, catalyzed by the enzyme Ras farnesyltransferase and thought to be essential for anchoring cytosolic Ras to the cell membrane and its resultant activation, has been, for at least a decade, the focus of intense research by several groups aiming to develop novel antitumor compounds via inhibition of this enzyme. Several farnesyltransferase inhibitors emanating from such efforts are currently reported to be undergoing clinical trials but some of these will suffer from serious drawbacks such as questionable efficacy in humans resulting from geranylgeranylation bypass and problems relating to target selectivity (D. M. Leonard “Ras Farnesyltransferase: A New Therapeutic Target” J. Med. Chem. 1997, 40, 2971-2990). Other groups have focussed their research efforts at developing Raf kinase inhibitors as anticancer agents (K. Lackey et al. “The Discovery of Potent cRaf1 Kinase Inhibitors” Bioorg. Med. Chem. Lett., 2000, 10, 223-226). Alternatively, within the framework of this protein signalling cascade, small molecule inhibitors of Ras/Raf interaction are viewed to be of great interest and potential as effective and novel mechanism based cancer chemotherapeutic agents.

### SUMMARY OF THE INVENTION

The object of the present invention is to provide new anticancer compounds (especially new Ras signalling pathway modulators) having high activity. It is another object of the present invention to provide suitable pharmaceutical compositions. Moreover, it is desired that these new compounds are capable of being utilized in the treatment of cancer (especially pancreatic, lung and colon cancers).

The present invention describes compounds, their pharmacologically acceptable salts, or solvates and hydrates, respectively, and formulations that are new and exhibit high activity and can be orally administered. The present invention furthermore relates to pro-drugs, optically active forms, racemates and diastereomers of such compounds and salts. These compounds and salts may, in turn, be pro-drugs which will be metabolically activated. The present invention furthermore describes pharmaceutical compositions containing said compounds and salts, respectively, as active ingredient. Furthermore, the use of such active ingredients in the treatment of cancer is disclosed.

The present invention provides compounds of Formula (1): 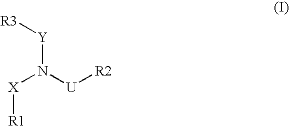
wherein
U is (CH₂)ₙ, CO, SO₂ or CONH;
n is 0, 1, 2, 3, 4 or 5;
X is CH₂, CO, SO₂ or CONH;
Y is CH₂, CO, SO₂ or CONH;
R1 is an optionally substituted aryl, aralkyl, heteroaryl or heteroarylalkyl;
R2 is an optionally substituted heteroalkyl, aryl, aralkyl, heteroaryl, heteroaralkyl, cycloalkyl, heterocycloalkyl or heteroalkylcycloalkyl and
R3 is an optionally substituted alkyl, alkenyl, alkinyl, heteroalkyl, cycloalkyl, alkylcycloalkyl, heterocycloalkyl, heteroalkylcycloalkyl, aryl, heteroaryl, heteroarylalkyl or aralkyl;
   or a pharmacologically acceptable salt, solvate, hydrate or formulation thereof.

It should be appreciated that certain compounds of Formula (I) or (II) may have tautomeric forms from which only one might be specifically mentioned or depicted in the following description, different geometrical isomers (which are usually denoted as cis/trans isomers or more generally as (E) and (Z) isomers) or different optical isomers as a result of one or more asymmetric or chiral carbon atoms (which are usually nomenclatured under the Cahn-Ingold-Prelog or R/S system). Further, some compounds may display polymorphism. All these tautomeric forms, geometrical or optical isomers (as well as racemates and diastereomers) and polymorphous forms are included in the invention.

The term alkyl refers to a saturated or unsaturated (i.e. alkenyl and alkinyl) straight or branched chain alkyl group, containing from one or two to ten carbon atoms, preferably from one or two to six carbon atoms, e.g. 1 or 2 to 4 carbon atoms, for example methyl, ethyl, propyl, iso-propyl, butyl, iso-butyl, tert.-butyl, n-hexyl, 2,2-dimethylbutyl, n-octyl; ethenyl (vinyl), propenyl, iso-propenyl, butenyl, isoprenyl or hexa-2-enyl; ethinyl, propinyl or butinyl groups.

The terms alkenyl and alkinyl refer to unsaturated straight or branched chain alkyl groups, containing from two to ten carbon atoms, preferably from two to six carbon atoms, e.g. 2 to 4 carbon atoms, for example ethenyl (vinyl), propenyl, iso-propenyl, butenyl, isoprenyl or hexa-2-enyl; ethinyl, propinyl or butinyl groups.

The term heteroalkyl refers to an alkyl, alkenyl or alkinyl group as defined herein where one or more carbon atoms are replaced by an oxygen, nitrogen, phosphorous or sulphur atom, for example an alkoxy group containing from one to ten carbon atoms, preferably from one to six carbon atoms, e.g. 1 to 4 carbon atoms, such as methoxy, ethoxy, propoxy, iso-propoxy, butoxy or tert.-butoxy; a (1-4C)alkoxy(1-4C)alkyl group such as methoxymethyl, ethoxymethyl, 1-methoxyethyl, 1-ethoxyethyl, 2-methoxyethyl or 2-ethoxyethyl; or a cyano group; or a 2,3-dioxyethyl group. The term heteroalkyl furthermore refers to a group derived from a carboxylic acid or carboxylic acid amide containing from one to ten carbon atoms, preferably from one to six carbon atoms, e.g. 1 to 4 carbon atoms, and may, for example, be acyl containing from one to ten carbon atoms, preferably from one to six carbon atoms, e.g. 1 to 4 carbon atoms, such as acetyl, propionyl, butyryl or pivaloyl; acyloxy containing from one to ten carbon atoms, preferably from one to six carbon atoms, e.g. 1 to 4 carbon atoms such as acetyloxy, propionyloxy, butyryloxy or pivaloyloxy; carboxyalkyl containing from one to ten carbon atoms, preferably from one to six carbon atoms, e.g. 1 to 4 carbon atoms such as carboxymethyl, carboxyethyl, carboxypropyl, carboxybutyl, carboxyalkyl ester containing from one to ten carbon atoms, preferably from one to six carbon atoms, e.g. 1 to 4 carbon atoms, such as carboxyalkyl methyl ester, carboxyalkyl ethyl ester, carboxyalkyl propyl ester, carboxyalkyl isopropyl ester, carboxyalkyl butyl ester or carboxyalkyl tert.-butyl ester, carboxyalkyl amide or alkylcarbamoyl such as N-(1-4C)alkylcarbamoyl or N,N′-(1-4C)dialkylcarbamoyl) containing from one to ten carbon atoms, preferably from one to six carbon atoms, e.g. 1 to 4 carbon atoms such as N-methylcarbamoyl, N-ethylcarbamoyl, N-propylcarbamoyl, N,N′-dimethylcarbamoyl, N-ethyl-N-methylcarbamoyl or N,N′-dipropylcarbamoyl, alkoxycarbonyl containing from one to ten carbon atoms, preferably from one to six carbon atoms, e.g. 1 to 4 carbon atoms, such as methoxycarbonyl, ethoxycarbonyl, propoxycarbonyl, isopropoxycarbonyl, butoxy- or tert.-butoxycarbonyl or alkoxycarbonyloxy containing from one to ten carbon atoms, preferably from one to six carbon atoms, e.g. 1 to 4 carbon atoms such as methoxycarbonyloxy, ethoxycarbonyloxy, propoxycarbonyloxy, isopropoxycarbonyloxy, butoxycarbonyloxy, tert.-butoxycarbonyloxy.

The term cycloalkyl refers to a saturated or partially unsaturated cyclic group, having one or more rings, formed by a skeleton that contains from three to 14 carbon atoms, preferably from three, four, five or six to nine or ten carbon atoms, for example cyclopropyl, cyclobutyl, cyclopentyl, cyclohexyl, tetralin, cyclopentenyl or cyclohex-2-enyl groups.

The term heterocycloalkyl refers to a cycloalkyl group as defined herein where one or more carbon atoms are replaced by one or more oxygen, nitrogen, phosphorous or sulphur atoms. Specific examples for heterocyclalkyl are piperidino, morpholino, N-methyl-piperazino or N-phenyl-piperazino groups.

The term aryl refers to an aromatic cyclic group, having one or more rings, formed by a skeleton that contains from five to 14 carbon atoms preferably from five or six to nine or ten carbon atoms, for example phenyl, inden or naphthyl groups. Specific examples are a benzyl, tolyl, phenethyl, biphenyl, xylyl, cumyl, 2-, 3- or 4-methoxyphenyl, 2-, 3- or 4-ethoxyphenyl, 4-carboxyphenyl alkyl or a 4-hydroxyphenyl group.

The term heteroaryl refers to an aryl group as defined herein where one or more carbon atoms are replaced by an oxygen, nitrogen, phosphorous or sulphur atom, for example 4-pyridyl, 2-imidazolyl, 3-pyrazolyl, quinolinyl, isoquinolinyl, pyrrolyl, oxazolyl, isoxazolyl, thiazolyl, isothiazolyl, 1,2,3-triazolyl, 1,2,4-triazolyl, oxadiazolyl, thiadiazolyl, indolyl, indazolyl, tetrazolyl, pyrazinyl, pyridinyl, pyrimidinyl and pyridazinyl groups.

The terms aralkyl and heteroarylalkyl refer to groups that comprise both aryl or, respectively, heteroaryl as well as alkyl, alkenyl, alkinyl and/or heteroalkyl (for example alkoxy groups in case of aralkyloxy) and/or cycloalkyl and/or heterocycloalkyl ring systems as defined herein. Examples of such groups are arylalkyl-, arylalkenyl-, arylalkinyl-, arylheteroalkyl-, arylheteroalkenyl-, arytheteroalkinyl-, heteroarylheteroalkyl-, heteroarylheteroalkenyl-, heteroarylheteroalkinyl-, arylcycloalkyl-, heteroarylcycloalkyl-, arylheterocycloalkyl-, heteroarylheterocycloalkyl-, arylcycloalkenyl-, heteroarylcycloalkenyl-, arylcycloalkinyl-, heteroarylcycloalkinyl-, arylheteroalkenyl-, heteroarylheteroalkenyl-, arylheteroalkinyl-, heteroarylheteroalkinyl-, heteroarylalkyl-, heteroalkenyl- and heteroarylakinyl-groups, wherein the cyclic groups can be saturated or once, twice or three-times unsaturated. Examples are the tetrahydroisoquinolinyl, benzyl, benzyloxy, 2- or 3-ethyl-indolyl or 4-methylpyridino groups.

The terms alkylcycloalkyl and heteroalkylcycloalkyl refer to groups that comprise both cycloalkyl or, respectively, heterocycloalkyl as well as alkyl, alkenyl, alkinyl and/or heteroalkyl (for example alkoxy groups in case of aralkyloxy) groups as defined herein. Examples of such groups are alkylcycloalkyl, alkenylcycloalkyl, alkinylcycloalkyl, alkylheterocycloalkyl, alkenylheterocycloalkyl, alkinylheterocycloalkyl, heteroalkylcycloalkyl, heteroalkenylcycloalkyl, heteroalkinylcycloalkyl, heteroalkylheterocycloalkyl, heteroalkenylheterocylcloalkyl, heteroalkinylheterocycloalkyl, which cyclic groups can be saturated or once, twice or three-times unsaturated.

Any alkyl, alkenyl, alkinyl, heteroalkyl, cycloalkyl, heterocycloalkyl, aryl, heteroaryl aralkyl or heteroarylalkyl groups as defined herein may be substituted with one or more halogen atoms, NH₂, SH, NO₂ or OH groups or unsubstituted alkyl, heteroalkyl, aryl, aralkyl, aralkyloxy, heteroaryl, cycloalkyl or heterocycloalkyl groups as defined herein.

The term “optionally substituted” refer to groups wherein one or more hydrogen atoms may be replaced a halogen atom, a NH₂, SH, NO₂ or OH group or by a unsubstituted alkyl, heteroalkyl, aryl, aralkyl, aralkyloxy, heteroaryl, cycloalkyl or heterocycloalkyl group as defined herein.

Preferred and/or advantageous embodiments of the invention are subject-matter of the subclaims.

Preferred are compounds of Formula (I), wherein U is (CH₂)ₙ and n is 0, 1 or 2.

Further preferred are compounds of Formula (1), wherein R1 is an optionally substituted phenyl ring; moreover preferred the phenyl ring is substituted by a benzyloxy group.

Moreover preferred are compounds of Formula (1), wherein R2 is heterocycloalkyl or heteroaryl (especially preferred nitrogen containing heterocycloalkyl or heteroaryl groups).

Further preferred are compounds of Formula (1), wherein R2 is a pyridyl or a piperidyl group.

Further preferred are compounds of Formula (I), wherein R3 is aryl or aralkyl.

Moreover preferred are compounds of formula (1), wherein R1 is a group of the formula 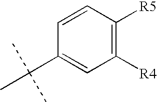
wherein R4 is H, alkyloxy or aralkyloxy (more preferred H, methoxy or benzyloxy) and R5 is F, Cl, alkyl, heteroalkyl, aryl, aralkyl, heteroaryl or heteroarylalkyl (preferred Cl, methoxy or benzyloxy; more preferred benzyloxy).

Further preferred are compounds of Formula (I) wherein R3 is a C₁-C₆ alkyl group (especially an isopropyl group).

Moreover preferred are compounds of Formula (I), wherein X is CO or SO₂ and Y is CH₂.

Further preferred are compounds of Formula (I), wherein X is CH₂ and Y is CO or SO₂.

Moreover preferred are compounds of Formula (II) 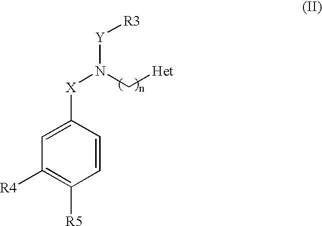
wherein Het is a pyridyl group; n is 0, 1 or 2; X is CH₂; Y is CO or SO₂; R3 is aryl or aralkyl; R4 is H, alkyloxy or aralkyloxy (more preferred H, methoxy or benzyloxy) and R5 is F, Cl, alkyl, heteroalkyl, aryl, aralkyl, heteroaryl or heteroarylalkyl (preferred Cl, methoxy or benzyloxy; more preferred benzyloxy).

Further preferred are compounds of Formula (II) wherein Het is a pyridyl group; n is 0, 1 or 2; X is CO or SO₂; Y is CH₂; R3 is aryl or aralkyl; R4 is H, alkyloxy or aralkyloxy (preferred H, methoxy or benzyloxy) and R5 is F, Cl, alkyl, heteroalkyl, aryl, aralkyl, heteroaryl or heteroarylalkyl (preferred Cl, methoxy or benzyloxy; more preferred benzyloxy).

Moreover preferred are compounds of Formula (II), wherein Het is a piperidyl group; n is 0, 1 or 2; X is CH₂; Y is CO or SO₂; R3 is aryl or aralkyl; R4 is H, alkyloxy or aralkyloxy (more preferred H, methoxy or benzyloxy) and R5 is F, Cl, alkyl, heteroalkyl, aryl, aralkyl, heteroaryl or heteroarylalkyl (preferred Cl, methoxy or benzyloxy; more preferred benzyloxy).

Further preferred are compounds of Formula (II) wherein Het is a piperidyl group; n is 0, 1 or 2; X is CO or SO₂; Y is CH₂; R3 is aryl or aralkyl; R4 is H, alkyloxy or aralkyloxy (preferred H, methoxy or benzyloxy) and R5 is F, Cl, alkyl, heteroalkyl, aryl, aralkyl, heteroaryl or heteroarylalkyl (preferred Cl, methoxy or benzyloxy; more preferred benzyloxy).

Further preferred are compounds of Formulas (I) or (II) wherein R3 is a group of the Formula (CH₂)ₘPh wherein m is 0, 1, 2, 3, 4 or 5 (more preferred m is 2, 3 or 4) and wherein the phenyl group may be optionally substituted.

Further preferred, the compound of Formula (I) or (II) is not N-(4-Benzyloxy-3-methoxybenzyl)-N-(2-pyridin-2-yl-ethyl)-2-chloro-benzamide.

Especially preferred are the following compounds:
N-(4-Benzyloxy-3-methoxy-benzyl)-N-(2-pyridin-4-yl-ethyl)-benzenesulfonamide,
N-(2-Chloro-benzyl)-N-(2-pyridin-2-yl-ethyl)-(4-benzyloxy-3-methoxy)-benzamide,
N-(4-Benzyloxy-3-methoxy-benzyl)-N-(2-piperidin-2-yl-ethyl)-4-phenyl-butyric acid amide,
N-(4-Benzyloxy-3-methoxy-benzyl)-N-(2-pyridin-2-yl-ethyl)-5-phenyl-pentanoic acid amide.

The present invention also relates to pharmacologically acceptable salts, or solvates and hydrates, respectively, and to compositions and formulations of compounds of Formula (I) or (II). The pharmaceutical compositions according to the present invention contain at least one compound of Formula (I) or (II) as the active agent and optionally carriers and/or diluents and/or adjuvants. Examples of such pharmacologically acceptable salts of sufficiently basic compounds of Formula (I) or (II) are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleinic and salicylic acid. Further, a sufficiently acid compound of Formula (I) or (II) may form alkali or earth alkaline metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, N-methyl-D-aminomethane (meglumin), piperidine, morpholine, tris-(2-hydroxyethyl)amine, lysine or arginine salts. Compounds of Formula (I) or (II) may be solvated, especially hydrated. The hydratisation can occur during the process of production or as a consequence of the hygroscopic nature of the initially water free compounds of Formula (I) or (II). The compounds of Formula (I) or (II) contain asymmetric C-atoms and may be present either as achiral compounds, mixtures of diastereomers, mixtures of enantiomers or as optically pure compounds.

The present invention also relates to pro-drugs which are composed of a compound of Formula (I) or (II) and at least one pharmacologically acceptable protective group which will be cleaved off under physiological conditions, such as an alkoxy-, aralkyloxy-, acyl- or acyloxy group as defined herein, e.g. ethoxy, benzyloxy, acetyl or acetyloxy.

As mentioned above, therapeutically useful agents that contain compounds of Formula (I) or (II), their solvates, salts and formulations are also comprised in the scope of the present invention. In general, compounds of Formula (I) or (II) will be administered by using the known and acceptable modes known in the art, either alone or in combination with any other therapeutic agent. Such therapeutically useful agents can be administered by one of the following routes: oral, e.g. as tablets, dragees, coated tablets, pills, semisolids, soft or hard capsules, for example soft and hard gelatine capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containg the active ingredient or intranasal. For the production of such tablets, pills, semisolids, coated tablets, dragees and hard, e.g. gelatine, capsules the therapeutically useful product may be mixed with pharmaceutically inert, inorganic or organic excipients as are e.g. lactose, sucrose, glucose, gelatin, malt, silica gel, starch or derivatives thereof, talc, stearinic acid or their salts, dried skim milk, and the like. For the production of soft capsules one may use excipients as are e.g. vegetable, petroleum, animal or synthetic oils, wax, fat, polyols. For the production of liquid solutions, emulsions or suspensions or syrups one may use excipients as are e.g. water, alcohols, aqueous saline, aqueous dextrose, polyols, glycerin, vegetable, petroleum, animal or synthetic oils. For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilisation, e.g. UV stabilizers, emulsifiers, sweetener, aromatisers, salts to change the osmotic pressure, buffers, coating additives and antioxidants.

Combinations with other therapeutic agents may include other therapeutically useful agents, e.g. that are used to prevent or treat cancer.

In the following the invention is described in more detail with reference to examples. These examples are intended for illustration only and are not to be construed as any limitation.

### EXAMPLES

Biological Assays:

### Determination of Activity in Secondary Luciferase Assays in Mammalian Cells.

Plasmids containing multiple copies of a serum response element (SRE) or Ap-1 response element upstream of luciferase were used to assess transcription induced by serum stimulation or transfection with a plasmid expressing RasV12. NIH3T3, CHO, or HEK293 cells were transiently transfected with response element-luciferase reporters, and serum starved. Compounds were added, followed by serum stimulation after one hour; 5 hours later, the cells were harvested with 1XCCLR reagent (Promega luciferase assay system, Promega, Madison, Wis., USA), and processed as advised by manufacturer. Alternatively, cells were co-transfected with reporter and RasV12 expression construct, starved, then treated with compound, and similarly assayed. Additionally, the SRE-luciferase construct was used to develop stable cell lines in a CHO cell background, and used in similar assays.

### Anchorage Independent Growth Versus Inhibition of Monolayer Growth.

NRK cells expressing activated K-Ras, or HT1080 cells with activated N-Ras, or PANC1 cells with activated K-Ras, or HCT116 cells with activated K-Ras, or A2058 with activated B-Raf (H. Davies et al. “Mutations of the BRAF Gene in Human Cancers” Nature, 2002, 417, 949-954) were used in standard assays for measurment of anchorage independent growth (J.-J. Yang et al. “Ras Signals to the Cell Cycle Machinery via Multiple Pathways to Induce Anchorage-Independent Growth” Mol. Cell. Biol. 1998, 18, 2586-2595). In parallel, comparable concentrations of compounds were assessed for inhibition of cell growth of the same cell lines cultured in monolayers.

Synthetic Methods:

### General Procedure for Solution-Phase Synthesis of Secondary Amines: N-(4-Benzyloxy-3-methoxy-benzyl)-N-(2-pyridin-2-yl-ethyl)-amine

A solution of 4-benzyloxy-3-methoxybenzaldehyde (10.0 g, 41.3 mmol) and 2-(2-aminoethyl)pyridine (5.3 mL, 44 mmol) in MeOH (170 mL) was stirred at room temperature for 5.5 h. NaBH₄ (2.5 g, 66 mmol) was now added carefully to the solution with ice cooling. After stirring for 1 h, the solution was quenched with 1 N aqueous NaOH and then concentrated under reduced pressure. An Et₂O extract of the residue was washed with 10% aqueous HCl, the aqueous phase was made alkaline with 10% aqueous NaOH, and then re-extracted with Et₂O. The organic phase was dried (anhydrous Na₂SO₄) and solvent was evaporated in vacuo to yield 14.0 g (97%) of the secondary amine as a viscous oil. MS (electrospray, +ions): m/z=349 (M+H).

When the product of this reaction contained a N-tert-butyloxycarbonyl (Boc) protected amine functionality, the acid wash in the reaction work-up was eliminated.

### General Procedure for Solution-Phase Parallel Synthesis of Amides (or Carbamates or Sulfonamides or Ureas) Utilizing an Acid Chloride (or Chloroformate or Sulfonyl Chloride or Isocyanate, Respectively) with Scavenger Resin Purification: N-(4-Benzyloxy-3-methoxy-benzyl)-N-(2-pyridin-2-yl-ethyl)-2-chloro-benzamide

A solution of N-(4-benzyloxy-3-methoxy-benzyl)-N-(2-pyridin-2-yl-ethyl)-amine (0.174 g, 0.500 mmol) in 1,2-dichloroethane (5 mL) was treated with polymer supported morpholine (0.400 g, 1.10 mmol) and 2-chlorobenzoyl chloride (0.076 mL, 0.60 mmol) and the resulting slurry was slowly stirred at room temperature for 24 h. Polymer supported isocyanate (0.150 g, 0.300 mmol) and polymer supported tris(2-aminoethyl)amine (0.500 g, 1.70 mmol) were added along with additional 1,2-dichloroethane (3 mL) and the mixture was stirred for an additional 24 h. The reaction mixture was filtered through a PTFE membrane filter and the resin bed was washed with CH₂Cl₂ (10 mL). The filtrate was stirred with saturated aqueous NaHCO₃ (5 mL) for 3 h and then with H₂O (5 mL) for 0.5 h. The organic phase was refiltered through a plug of anhydrous Na₂SO₄ and solvent was evaporated to yield 0.205 g (84%) of the title compound. MS (electrospray, +ions): m/z=487 (M+H).

For reactions utilizing isocyanates, polymer supported morpholine was excluded.

### General Procedure for Solution-Phase Parallel Synthesis of Amides Utilizing an Acid, with Aqueous Work-Up: N-(4-Benzyloxy-3-methoxy-benzyl)-N-(2-pyridin-2-yl-ethyl)-5-phenyl-pentanoic acid amide

A solution of N-(4-benzyloxy-3-methoxy-benzyl)-N-(2-pyridin-2-yl-ethyl)-amine (0.174 g, 0.500 mmol), HOBt (0.074 g, 0.55 mmol), EDCI methiodide (0.163 g, 0.550 mmol) and 5-phenylvaleric acid (0.107 g, 0.600 mmol) in CH₃CN-DMF (3:1, 4 mL) was shaken at room temperature for 48 h. After removal of solvent under reduced pressure, the residue was dissolved in EtOAc (15 mL). The organic phase was sequentially washed with H₂O (3×3 mL), saturated aqueous NaHCO₃ (3×3 mL), H₂O (3 mL), brine (3 mL) and dried over anhydrous Na₂SO₄. Solvent was evaporated and the residue was redissolved in CH₂Cl₂ (5 mL) and stirred with polymer supported isocyanate (0.300 g, 0.600 mmol) for 24 h. The resin was removed by filtering the slurry through a PTFE membrane filter and the resin bed was washed with CH₂Cl₂ (10 mL). Evaporation of the filtrate afforded 0.220 g (87%) of the title compound. MS (electrospray, +ions): m/z 509(M+H).

### General Procedure for Solution-Phase Parallel Synthesis of Secondary Amines: N-(4-Benzyloxy-benzyl)-N-(2-pyridin-2-yl-ethyl)-amine

A solution of 4-benzyloxy benzaldehyde (0.212 g, 1.00 mmol) and 2-(2-aminoethyl)pyridine (0.179 mL, 1.50 mmol) in MeOH (3 mL) was stirred at room temperature for 14 h. Polymer supported borohydride (0.800 g, 2.00 mmol) was added and the reaction mixture was stirred at room temperature for 20 h. Polymer supported 4-benzyloxybenzaldehyde (0.413 g, 1.00 mmol), polymer supported tris(2-aminoethyl)amine (0.300 g, 1.00 mmol) and CH₂Cl₂ (3 mL) were now added and the reaction mixture was stirred for an additional 20 h. The reaction mixture was filtered and the resin bed was washed with CH₂Cl₂ (10 mL). The filtrate was evaporated and the residue was dissolved in 20% AcOH in MeOH (4 mL) and subjected to ion-exchange chromatography (Varian SCX column, 2 g, 0.79 meq/g). After washing with MeOH, the product was eluted off the column with 2 N NH₃ in MeOH (12 mL). Subsequent evaporation of the eluate afforded 0.275 g (86%) of the title compound. MS (electrospray, +ions): m/z=319 (M+H).

### General Procedure for Solution-Phase Parallel Synthesis of Amides Using an Acid, with Ion-Exchange Purification: N-(4-Benzyloxy-benzyl)-N-(2-pyridin-2-yl-ethyl)-4-phenyl-butyric acid amide

A solution of N-(4-benzyloxy-benzyl)-N-(2-pyridin-2-yl-ethyl)-amine (0.140 g, 0.440 mmol), HOBt (0.074 g, 0.55 mmol), EDCI methiodide (0.163 g, 0.550 mmol) and 4-phenylbutyric acid (0.090 g, 0.55 mmol) in CH₃CN-DMF (3:1, 4 mL) was shaken at room temperature for 48 h. Solvent was evaporated and the residue was dissolved in 20% AcOH in MeOH (4 mL) and subjected to ion-exchange chromatography (Varian SCX column, 2 g, 0.79 meq/g). After washing with MeOH, the product was eluted off the column with 2 N NH₃/MeOH (12 mL). The eluate was evaporated and the residue was redissolved in CH₂Cl₂ (5 mL) and stirred with polymer supported isocyanate (0.200 g, 0.400 mmol) for 24 h. The resin was removed by filtering the slurry through a PTFE membrane filter and the resin bed was washed with CH₂Cl₂. Evaporation of the filtrate afforded 0.168 g (82%) of the title compound. MS (electrospray, +ions): m/z=465 (M+H).

With regards to the general synthetic protocols described above, where necessary, free carboxylic acid or free amino functionalized reagents were utilized as their methyl ester or N-tert-butyloxycarbonyl (Boc) protected forms, respectively. Regeneration of the free acid or free amine from the corresponding protected form was accomplished utilizing standard protocols. 5-Phenylpentanoyl chloride was prepared from the commercially available 5-phenylvaleric acid utilizing standard methodology (oxalyl chloride in CH₂Cl₂)-

### 2-[N-(4-Benzyloxy-3-methoxy-benzyl)-N-(2-pyridin-2-yl-ethyl)-amino]-1-phenyl-ethanol

A solution of N-(4-benzyloxy-3-methoxy-benzyl)-N-(2-pyridin-2-yl-ethyl)-amine (0.17 g, 0.50 mmol) in methanol (5 mL) was treated with 1,2-epoxyethylbenzene (0.072 g, 0.60 mmol) and the resulting mixture was stirred at 60° C. for 24 h. The reaction mixture was concentrated under reduced pressure and the residue was dissolved in 1,2-dichloroethane (5 mL). To this solution polymer supported isocyanate (0.15 g, 0.30 mmol) and polymer supported tris(2-aminoethyl)amine (0.50 g, 1.7 mmol) were added and the mixture was stirred for an additional 24 h. The reaction mixture was filtered through a PTFE membrane filter and the resin bed was washed with CH₂Cl₂ (3×3 mL). The organic phase was refiltered through a plug of anhydrous Na₂SO₄ and solvent was evaporated to yield 0.19 g (80%) of the title compound. MS (electrospray, +ions): m/z=469 (M+H).

### N-(4-Benzyloxy-3-hydroxmethyl-benzyl)-N-(2-pyridin-2-yl-ethyl)-5-phenyl-pentanoic acid amide

### Step A: (2-Benzyloxy-5-formyl)-benzoic acid benzyl ester

A mixture of 5-formylsalicylic acid (5.6 g, 34 mmol), potassium carbonate (5.5 g, 40 mmol), benzyl bromide (6.9 g, 40 mmol) and 18-crown-6 (0.4 g) in acetone (100 mL) was stirred and heated at reflux overnight. After filtration, the filtrate was concentrated under reduced pressure and the residue was redissolved in EtOAc. The organic phase was washed with 1N aqueous NaOH, brine and dried over anhydrous Na₂SO₄. After solvent was evaporated, the residual pale yellow solid was washed with 5% Et₂O/hexane to provide 9.0 g (77%) of the product.

### Step B: N-(4-Benzyloxy-3-benzyloxycarbonyl-benzyl)-N-(2-pyridin-2-yl-ethyl)-amine

A mixture of (2-benzyloxy-5-formyl)-benzoic acid benzyl ester (1.0 g, 2.9 mmol), 2-(2-aminoethyl)pyridine (0.353 g, 2.89 mmol) and NaB(OAc)₃H (0.858 g, 4.05 mmol) in 1,2-dichloroethane (10 mL) was stirred at room temperature for 24 h. The reaction mixture was quenched with saturated aqueous NaHCO₃ (10 mL) and extracted with EtOAc. The organic extract was washed with brine, dried (anhydrous Na₂SO₄) and solvent was evaporated to provide 1.2 g (92%) of the product.

### Step C: N-(4-Benzyloxy-3-hydroxymethyl-benzyl)-N-(2-pyridin-2-yl-ethyl)-amine

A solution of N-(4-benzyloxy-3-benzyloxycarbonyl-benzyl)-N-(2-pyridin-2-yl-ethyl)-amine (1.28 g, 2.83 mmol) in THF (5 mL) was added dropwise to a suspension of LiAlH₄ (0.279 g, 7.37 mmol) in THF (5 mL) at room temperature. The resulting mixture was stirred and heated at reflux for 24 h, cooled to 0° C., and quenched with water (1.3 mL) and 10% aqueous NaOH (1.3 mL). The mixture was filtered and the residual solid was washed with EtOAc. The organic phase was washed with brine, dried (anhydrous Na₂SO₄) and the solvent was evaporated. The residual oil was purified by silica gel column chromatography to give 0.51 g (52%) of the product.

### Step D: N-(4-Benzyloxy-3-hydroxymethyl-benzyl)-N-(2-pyridin-2-yl-ethyl)-5-phenyl-pentanoic acid amide

To a stirred solution of N-(4-benzyloxy-3-hydroxymethyl-benzyl)-N-(2-pyridin-2-yl-ethyl)-amine (0.51 g, 1.5 mmol), Et₃N (0.62 mL, 4.4 mmol) and a small amount of DMF in CH₂Cl₂ (15 mL), a cooled solution (0° C.) of 5-phenylpentanoyl chloride (0.316 g, 1.61 mmol) in CH₂Cl₂ (1 mL) was added dropwise. The resulting mixture was stirred at 0° C. for 45 min. and at room temperature for 1 h. The solvent was concentrated under reduced pressure and the residue was redissolved in EtOAc. The organic phase was washed with brine, dried (anhydrous Na₂SO₄) and the solvent was evaporated. The residual oil was purified by silica gel column chromatography to yield 0.26 g (35%) of the title compound. MS (electrospray, +ions): m/z=509 (M+H).

### N-(4-Benzyloxy-3-dimethylaminomethyl-benzyl)-N-(2-pyridin-2-yl-ethyl)-5-phenyl-pentanoic acid amide

### Step A: N-(4-Benzyloxy-3-methanesulfonyloxymethyl-benzyl)-N-(2-pyridin-2-yl-ethyl)-5-phenyl-pentanoic acid amide

A mixture of N-(4-benzyloxy-3-hydroxymethyl-benzyl)-N-(2-pyridin-2-yl-ethyl)-5-phenyl-pentanoic acid amide (4.0 g, 7.9 mmol) and Et₃N (0.80 g, 7.9 mmol) in CH₂Cl₂ (100 mL) was stirred and cooled to 0° C. Methanesulfonyl chloride (0.91 g, 8.0 mmol) was added slowly and after addition was complete, the mixture was allowed to warn to room temperature and stirred for 3 h. The reaction was quenched with water (50 mL), the organic layer was washed with brine, dried (anhydrous Na₂SO₄) and the solvent was evaporated. The residue was subjected to silica gel column chromatography to yield 0.46 g (10%) of the product.

### Step B: N-(4-Benzyloxy-3-dimethylaminomethyl-benzyl)-N-(2-pyridin-2-yl-ethyl)-5-phenyl-pentanoic acid amide

A mixture of N-(4-benzyloxy-3-methanesulfonyloxymethyl-benzyl)-N-(2-pyridin-2-yl-ethyl)-5-phenyl-pentanoic acid amide (0.46 g, 0.78 mmol) and potassium carbonate (0.11 g, 0.80 mmol) in acetone (20 mL) was stirred and treated with a slow stream of Me₂NH_{(g)} and the mixture was heated at reflux for 30 min. The solvent was concentrated and the residue was purified by silica gel column chromatography to yield 0.26 g (62%) of the title compound. MS (electrospray, +ions): m/z=536 (M+H).

### N-(4-Benzyloxy-3-carboxymethoxy-benzyl)-N-(2-pyridin-2-yl-ethyl)-5-phenyl-pentanoic acid amide

### Step A: (4-Benzyloxy-3-tert-butyloxycarbonylmethoxy)-benzaldehyde

A mixture of 4-benzyloxy-3-hydroxybenzaldehyde (5.0 g, 22 mmol), 18-crown-6 (0.58 g, 2.2 mmol) and K₂CO₃ (6.0 g, 43 mmol) in DMF (30 mL) was heated at 80-90° C. for 30 min. tert-Butyl bromoacetate (5.35 g, 27.4 mmol) was added and the resulting mixture was stirred and heated at 80-90° C. for 1 h. After cooling to room temperature, the reaction mixture was poured into ice-water (100 mL) and the resulting pale brown precipitate was collected and washed with 10% aqueous NaOH and water to yield 7.0 g (93%) of the product.

### Step B: N-(4-Benzyloxy-3-tert-butyloxycarbonylmethoxy-benzyl)-N-(2-pyridin-2-yl-ethyl)-amine

To a stirred solution of (4-benzyloxy-3-tert-butyloxycarbonylmethoxy)-benzaldehyde (4.0 g, 12 mmol) and 2-(2-aminoethyl)pyridine (1.57 g, 12.8 mmol) in 1,2-dichloroethane (50 mL), NaB(OAc)₃H (3.8 g, 18 mmol) was added at room temperature. The resulting mixture was stirred overnight, quenched with saturated aqueous NaHCO₃ (30 mL) and extracted with EtOAc. The organic phase was separated, washed with brine, dried (anhydrous Na₂SO₄) and solvent was evaporated. The resulting brown oil was purified by silica gel chromatography to provide 5.0 g (95%) of the product as an off-white powder.

### Step C: N-(4-Benzyloxy-3-tert-butyloxycarbonylmethoxy-benzyl)-N-(2-pyridin-2-yl-ethyl)-5-phenyl-pentanoic acid amide

To a stirred solution of N-(4-benzyloxy-3-tert-butyloxycarbonylmethoxy-benzyl)-N-(2-pyridin-2-yl-ethyl)-amine (6.1 g, 14 mmol) and Et₃N (5.7 mL, 41 mmol) in CH₂Cl₂ (30 mL), a solution of 5-phenylpentanoyl chloride (2.95 g, 15.0 mmol) in CH₂Cl₂ (8 mL) was added dropwise at 0° C. over 10 min. The resulting mixture was stirred at 0° C. for 1 h and at room temperature overnight. After removal of solvent, EtOAc (50 mL) was added to the residue and the organic phase was washed with water, 10% aqueous NaOH, brine and dried over anhydrous Na₂SO₄. After solvent evaporation, the resulting brown oil was purified by silica gel chromatography to provide 7.6 g (92%) of the product as a brown oil.

### Step D: N-(4-Benzyloxy-3-carboxymethoxy-benzyl)-N-(2-pyridin-2-yl-ethyl)-5-phenyl-pentanoic acid amide

To a stirred solution of N-(4-benzyloxy-3-tert-butyloxycarbonylmethoxy-benzyl)-N-(2-pyridin-2-yl-ethyl)-5-phenyl-pentanoic acid amide (7.33 g, 12.0 mmol) in CH₂Cl₂ (50 mL), TFA (12 mL) was added and the resulting mixture was stirred at room temperature overnight. Solvent was evaporated under reduced pressure, water (50 mL) was added and the pH was rendered alkaline with 10% aqueous NaOH. Careful neutralization with dilute AcOH resulted in an oily material which was extracted into EtOAc, washed with brine and dried over anhydrous Na₂SO₄. Solvent evaporation under reduced pressure yielded 5.43 g (82%) of the title compound as a viscous brown material. MS (electrospray, + ions): m/z=553 (M+H).

### N-(4-Benzyloxy-3-carbamoylmethoxy-benzyl)-N-(2-pyridin-2-yl-ethyl)-5-phenyl-pentanoic acid amide

To a stirred solution of N-(4-benzyloxy-3-carboxymethoxy-benzyl)-N-(2-pyridin-2-yl-ethyl)-5-phenyl-pentanoic acid amide (1.0 g, 1.8 mmol) in CH₂Cl₂ (10 mL) at room temperature, a solution of oxalyl chloride (0.174 mL, 1.99 mmol) in CH₂Cl₂ (0.5 mL) was added dropwise followed by a small amount of DMF. The resulting mixture was stirred at room temperature for 1 h and then added dropwise to conc. NH₄OH (20 mL) at −5° C. over 15 min to afford a white suspension. Solvent was evaporated and the residue was extracted into EtOAc. The organic phase was washed with brine, dried (anhydrous Na₂SO₄) and evaporated under reduced pressure to yield a pale brown oil, which was purified by flash silica gel chromatography (9:1 EtOAc:MeOH) to give 0.50 g (50%) of the title compound as a white powder. MS (electrospray, +ions): m/z=552 (M+H).

### N-(4-Hydroxy-3-methoxy-benzyl)-N-(2-pyridin-2-yl-ethyl)-5-phenyl-pentanoic acid amide

To a solution of N-(4-benzyloxy-3-methoxy-benzyl)-N-(2-pyridin-2-yl-ethyl)-5-phenyl-pentanoic acid amide (7.0 g, 1.4 mmol) in MeOH (200 mL), 5% Pd/C (0.700 g) was carefully added and the resulting mixture was subjected to hydrogenation at 50 psi overnight. The catalyst was filtered off and the filtrate was concentrated in vacuo to provide 5.6 g (97%) of the title compound.

### N-[3-Methoxy-4-(piperidin-1-yl-methoxy)-benzyl]-N-(2-pyridin-2-yl-ethyl)-5-phenyl-pentanoic acid amide

A mixture of N-(4-hydroxy-3-methoxy-benzyl)-N-(2-pyridin-2-yl-ethyl)-5-phenyl-pentanoic acid amide (1.0 g, 2.4 mmol), N-chloromethylpiperidine (0.35 g, 2.6 mmol), 18-crown-6 (0.070 g, 0.27 mmol) and K₂CO₃ (0.99 g, 7.2 mmol) in DMF (6 mL) was heated at 80-90° C. for 6 h. The reaction mixture was cooled to room temperature and diluted with EtOAc (50 mL). The organic layer was washed with brine, dried (anhydrous Na₂SO₄) and the solvent was evaporated under reduced pressure. The residue was purified by flash silica gel chromatography (9:1 EtOAc:MeOH) to provide 0.30 g (24%) of the title compound. MS (electrospray, +ions): m/z=516 (M+H).

### N-[3-Methoxy-4-(morpholin-1-yl-methoxy)-benzyl]-N-(2-pyridin-2-yl-ethyl)-5-phenyl-pentanoic acid amide

A mixture of N-(4-hydroxy-3-methoxy-benzyl)-N-(2-pyridin-2-yl-ethyl)-5-phenyl-pentanoic acid amide (1.0 g, 2.4 mmol), N-chloromethylmorpholine (0.39 g, 2.8 mmol), 18-crown-6 (0.076 g, 0.29 mmol) and K₂CO₃ (1.18 g, 8.54 mmol) in DMF (5 mL) was heated at 80-90° C. overnight. Inorganic material was filtered off and the filtrate was diluted with EtOAc (60 mL), washed with brine and dried over anhydrous Na₂SO₄. The volatiles were removed under reduced pressure and the residue was purified by flash silica gel chromatography (9:1 EtOAc:MeOH) to yield 0.24 g (19%) of the title compound. MS (electrospray, +ions): m/z=518 (M+H).

### N-(4-Benzoyloxy-3-methoxy-benzyl)-N-(2-pyridin-2-yl-ethyl)-5-phenyl-pentanoic acid amide

A solution of N-(4-hydroxy-3-methoxy-benzyl)-N-(2-pyridin-2-yl-ethyl)-5-phenyl-pentanoic acid amide (0.50 g, 1.2 mmol) and Et₃N (0.12 g, 1.2 mmol) in CH₂Cl₂ (10 mL) was stirred and cooled to 0° C. Benzoyl chloride (0.17 g, 1.2 mmol) was added dropwise and the mixture was warmed to room temperature and stirred for 2-3 h. The solvent was evaporated under reduced pressure and the residue was purified by silica gel column chromatography to provide 0.24 g (38%) of the title compound as an oil. MS (electrospray, +ions): m/z=523 (M+H).

### N-(4-Benzyloxy-3-methoxy-benzyl)-N-{2-[N′-(4-chlorophenyl)-N″-methoxy-guanidino]-ethyl}-isobutyric acid amide

### Step A: N-(4-Benzyloxy-3-methoxy-benzyl)-N-(2-tert-butyloxycarbamoyl-ethyl)-amine

To a stirred solution of 4-benzyloxy-3-methoxybenzaldehyde (8.0 g, 33 mmol) and N-Boc ethylenediamine (8.0 g, 50 mmol) in CH₂Cl₂ (100 mL) at room temperature, NaB(OAc)₃H (11 g, 45 mmol) was added and the resulting mixture was stirred overnight. The reaction was quenched with saturated aqueous NaHCO₃ (60 mL) and extracted with EtOAc (150 mL). The organic phase was washed with brine, dried (anhydrous Na₂SO₄) and the solvent was evaporated. The residue was purified by silica gel column chromatography to provide 5.9 g (46%) of the product as a white solid.

### Step B: N-(4-Benzyloxy-3-methoxy-benzyl)-N-(2-tert-butyloxycarbamoyl-ethyl)-isobutyric acid amide

To a solution of N-(4-benzyloxy-3-methoxy-benzyl)-N-(2-tert-butyloxycarbamoyl-ethyl)-amine (5.85 g, 15.1 mmol) and triethylamine (6.33 mL, 45.0 mmol) in CH₂Cl₂ (30 mL) at 0-5° C., a solution of isobutyryl chloride (1.77 g, 16.6 mmol) in CH₂Cl₂ (8 mL) was added dropwise over 50 min. The reaction mixture was stirred at 0° C. for 70 min, then at room temperature overnight. Solvent was evaporated under reduced pressure and EtOAc (50 mL) was added to the residue. The organic phase was washed with water (40 mL), 1N aqueous NaOH (20 mL), brine and dried over anhydrous Na₂SO₄. Solvent was evaporated and the crude product was purified by silica gel column chromatography (40% EtOAc/hexane) to yield 6.18 g (90%) of the product.

### Step C: N-(2-Amino-ethyl)-N-(4-benzyloxy-3-methoxy-benzyl)-isobutyric acid amide, trifluoroacetic acid salt

To a solution of N-(4-benzyloxy-3-methoxy-benzyl)-N-(2-tert-butyloxycarbamoyl-ethyl)-isobutyric acid amide (6.07 g, 13.3 mmol) in CH₂Cl₂ (60 mL) at 0° C., trifluoroacetic acid (10 mL) was added and the mixture was stirred at 0° C. for 1 h and at room temperature for 1.5 h. Solvent evaporation gave the crude product (6.46 g) as a yellow oil.

### Step D: N-(4-Benzyloxy-3-methoxy-benzyl)-N-{2-[3-(4-chlorophenyl)-thioureido]-ethyl}-isobutyric acid amide

To a stirred solution of 4-chlorophenyl isothiocyanate (1.04 g, 6.13 mmol) in CH₂Cl₂ (15 mL) under nitrogen, a solution of the above salt (1.98 g, 4.21 mmol) and triethylamine (0.78 mL, 5.6 mmol) in CH₂Cl₂ (15 mL) was added dropwise. The mixture was stirred at room temperature overnight and subsequently heated at 45-55° C. for 4 h. Solvent was evaporated and the residue was purified by silica gel column chromatography to afford 0.75 g (34%) of the product as a white solid.

### Step E: N-(4-Benzyloxy-3-methoxy-benzyl)-N-{2-[N-(4-chlorophenyl)-N″-methoxy-guanidino]-ethyl}-isobutyric acid amide

A mixture of N-(4-benzyloxy-3-methoxy-benzyl)-N-{2-[3-(4-chlorophenyl)-thioureido]-ethyl}-isobutyric acid amide (0.41 g, 0.79 mmol), mercury (II) oxide (0.17 g, 0.79 mmol), methoxylamine hydrochloride (66 mg, 0.79 mmol) and Et₃N (3 mL) was stirred at room temperature for 24 h. Et₂O (3 mL) was added and the reaction mixture was stirred at room temperature for an additional 24 h. Additional Et₂O was added and the reaction mixture was filtered through a pad of Celite®. The filtrate was evaporated and the residue was purified by silica gel column chromatography (60% EtOAc/hexane) to provide 0.14 g (32%) of the title compound as a white solid. MS (electrospray, +ions): m/z=539 (M+).

### N-(4-Benzyloxy-3-hydroxy-benzyl)-N-(2-pyridin-2-yl-ethyl)-2-chloro-benzamide

### Step A: 3-Acetyloxy-4-benzyloxybenzaldehyde

A solution of 3,4-dihydroxybenzaldehyde (5.52 g, 40.0 mmol) in acetone (120 mL) was treated with K₂CO₃ (16.6 g, 120 mmol) and benzyl bromide (7.18 g, 42.0 mmol) and the resulting mixture was stirred at room temperature for 24 h. Additional K₂CO₃ (8.3 g, 60 mmol) and acetyl chloride (6.28 g, 80.5 mmol) were now added and the mixture was stirred at room temperature for another 20 h. The reaction mixture was concentrated under reduced pressure, water (100 mL) was added to the residue which was then extracted with Et₂O (3×50 mL). The combined organic extract was washed with water (30 mL), dried (anhydrous Na₂SO₄), solvent was evaporated in vacuo and the residue was purified by silica gel column chromatography (10% EtOAc/hexane) to yield 7.80 g (72%, 2 steps) of the product as a white solid. MS (electrospray, +ions): m/z=271 (M+H).

### Step B: N-(4-Benzyloxy-3-hydroxy-benzyl)-N-(2-pyridin-2-yl-ethyl)-2-chloro-benzamide

To a solution of 3-acetyloxy-4-benzyloxybenzaldehyde (2.89 g, 10.7 mmol) in MeOH (20 mL), 2-(2-aminoethyl)pyridine (1.40 g, 11.4 mmol) was added at room temperature. The resulting mixture was stirred at room temperature for 5 h and then cooled to 0° C. To the cooled solution, NaBH₄ (0.647 g, 17.1 mmol) was added and the resulting mixture was stirred at 0° C. for 30 min and at room temperature for 1 h. The reaction mixture was concentrated under reduced pressure and the residue was suspended in water (20 mL) and quenched with acetic acid (2 mL). The aqueous solution was neutralized to pH 7 with solid NaHCO₃, then extracted with CH₂Cl₂ (3×30 mL). The combined organic extract was washed with H₂O (20 mL), brine (20 mL), dried (anhydrous Na₂SO₄) and the solvent was evaporated in vacuo to yield the crude secondary amine. To a solution of the above secondary amine in 1,2-dichloroethane (50 mL) at 0° C., triethylamine (4.26 mL, 30.6 mmol) and 2-chlorobenzoylchloride (1.62 mL, 12.8 mmol) were added dropwise. The resulting mixture was allowed to warm to room temperature, stirred for 16 h and then concentrated under reduced pressure. The residue was redissolved in a solution of MeOH (90 mL) and H₂O (10 mL) containing NaHCO₃ (3.36 g, 40.0 mmol). The resulting mixture was stirred at 80° C. for 2 h. After cooling to room temperature, the reaction mixture was concentrated under reduced pressure and co-evaporated with ethanol. The crude product was purified by silica gel column chromatography (1.5% MeOH/CH₂Cl₂) to yield 3.73 g (74%, 3 steps) of the title compound as a pale yellow solid. MS (electrospray, +ions): m/z=473 (M+H).

### N-(4-Benzyloxy-3-carbamoylmethoxy-benzyl)-N-(2-pyridin-2-yl-ethyl)-2-chloro-benzamide

### Step A: N-(4-Benzyloxy-3-methoxycarbonylmethoxy-benzyl)-N-(2-pyridin-2-yl-ethyl)-2-chloro-benzamide

A solution of N-(4-benzyloxy-3-hydroxy-benzyl)-N-(2-pyridin-2-yl-ethyl)-2-chloro-benzamide (1.00 g, 2.11 mmol) in acetone (10 mL) was treated with K₂CO₃ (0.874 g, 6.33 mmol) and methyl bromoacetate (0.355 g, 2.32 mmol) and the resulting mixture was stirred at room temperature for 16 h. Solvent was evaporated under reduced pressure, the residue was redissolved in CH₂Cl₂ (50 mL), washed with H₂O (2×10 mL), brine (10 mL) and dried over anhydrous Na₂SO₄. The solvent was evaporated under reduced pressure to provide the crude product (1.39 g) as a brown yellow oil. MS (electrospray, + ions): m/z=546 (M+H). This crude product was pure enough for the next step.

### Step B: N-(4-Benzyloxy-3-carbamoylmethoxy-benzyl)-N-(2-pyridin-2-yl-ethyl)-2-chloro-benzamide

A solution of N-(4-benzyloxy-3-methoxycarbonylmethoxy-benzyl)-N-(2-pyridin-2-yl-ethyl)-2-chloro-benzamide (0.14 g, 0.25 mmol) in MeOH (0.7 mL) was treated with NH₄Cl (0.05 g, 1 mmol) and conc. NH₄OH (0.5 mL) and the resulting mixture was stirred at room temperature for 16 h. After solvent evaporation, the residue was purified by silica gel column chromatography (40:1 CH₂Cl₂:MeOH) to yield 0.059 g (45%, 2 steps) of the title compound as an oil. MS (electrospray, +ions): m/z=531 (M+H).

### N-[4-Benzyloxy-3-(2-hydroxy-ethoxy)-benzyl]-N-(2-pyridin-2-yl-ethyl)-2-chloro-benzamide

A solution of N-(4-benzyloxy-3-hydroxy-benzyl)-N-(2-pyridin-2-yl-ethyl)-2-chloro-benzamide (0.24 g, 0.50 mmol) in acetone (4 mL) was treated with K₂CO₃ (0.55 g, 4.0 mmol) and 2-bromoethanol (0.26 g, 2.0 mmol). The resulting mixture was stirred at 60° C. for 48 h and concentrated under reduced pressure. The residue was dissolved in CH₂Cl₂ (15 mL), washed with H₂O (5 mL), saturated aqueous NaHCO₃ (5 mL), brine (5 mL) and dried over anhydrous Na₂SO₄. The solvent was evaporated under reduced pressure and the residue was purified by silica gel column chromatography (40:1 CH₂Cl₂:MeOH) to yield 0.12 g (54%) of the title compound as an oil. MS (electrospray, +ions): m/z=518 (M+H).

### N-[4-Benzyloxy-3-(2-dimethylamino-ethoxy)-benzyl]-N-(2-pyridin-2-yl-ethyl)-2-chloro-benzamide

A solution of N-(4-benzyloxy-3-hydroxy-benzyl)-N-(2-pyridin-2-yl-ethyl)-2-chloro-benzamide (0.24 g, 0.50 mmol) in DMF (3 mL) was treated with K₂CO₃ (0.69 g, 5.0 mmol) and 2-dimethylaminoethylchloride HCl salt (0.14 g, 1.0 mmol). The resulting mixture was stirred at room temperature for 17 h and then concentrated under reduced pressure. The residue was redissolved in CH₂Cl₂ (15 mL), washed with H₂O (5 mL), saturated aqueous NaHCO₃ (5 mL), brine (5 mL) and dried over anhydrous Na₂SO₄. The solvent was evaporated under reduced pressure and the residue was purified by silica gel column chromatography (20:1 CH₂Cl₂:MeOH) to yield 0.19 g (70%) of the title compound as an oil. MS (electrospray, +ions): m/z=545 (M+H).

### N-(4-Benzyloxy-3-dimethylcarbamoylmethoxy-benzyl)-N-(2-pyridin-2-yl-ethyl)-2-chloro-benzamide

A solution of N-(4-benzyloxy-3-hydroxy-benzyl)-N-(2-pyridin-2-yl-ethyl)-2-chloro-benzamide (0.24 g, 0.50 mmol) in DMF (3 mL) was treated with K₂CO₃ (0.35 g, 2.5 mmol) and 2-chloro-N,N-dimethylacetamide (0.12 g, 1.0 mmol). The resulting mixture was stirred at room temperature for 16 h and then concentrated under reduced pressure. The residue was redissolved in CH₂Cl₂ (15 mL), washed with H₂O (2×5 mL), saturated aqueous NaHCO₃ (5 mL), brine (5 mL) and dried over anhydrous Na₂SO₄. The solvent was evaporated in vacuo and the residue was purified by silica gel column chromatography (20:1 CH₂Cl₂:MeOH) to yield 0.21 g (77%) of the title compound as an oil. MS (electrospray, +ions): m/z=559 (M+H).

The following compounds were synthesized according to the procedures described above. All compounds were identified by electrospray mass spectroscopy and tested in the assays described above. They all show IC₅₀'s in between 5 and 60 μM range in luciferase assays. Some of the compounds inhibited colony formation in the anchorage independent growth assay in the cell lines mentioned above at 4 μM concentration and anchorage dependent growth in the same cells at 1 μM concentration

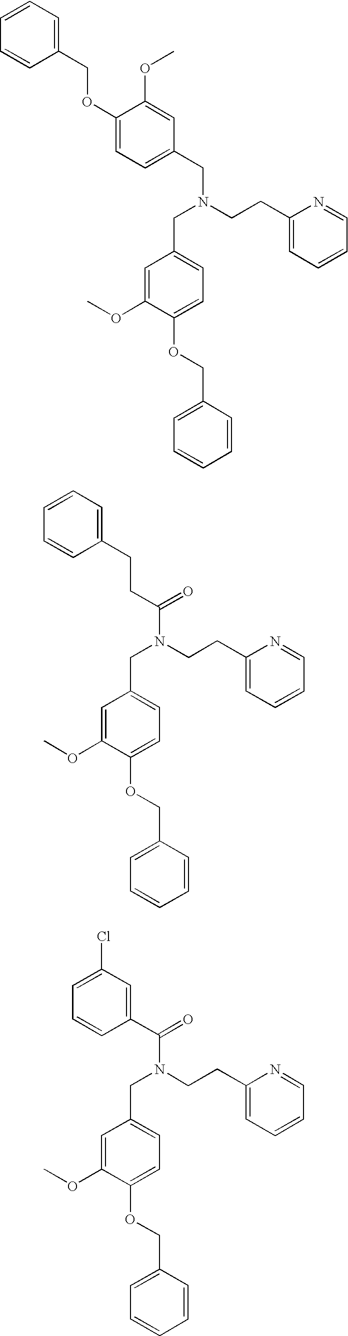

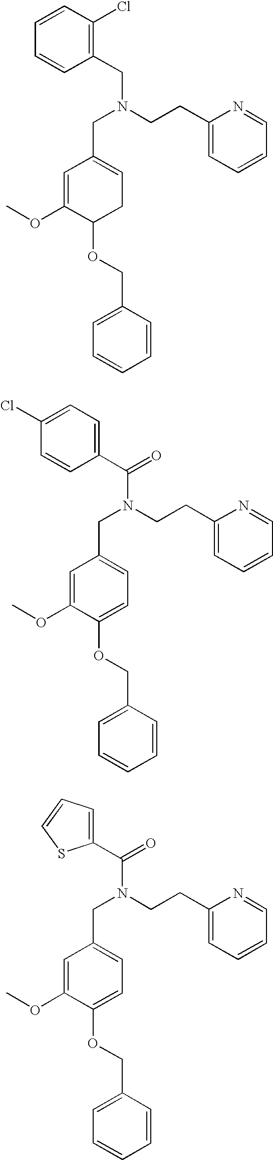

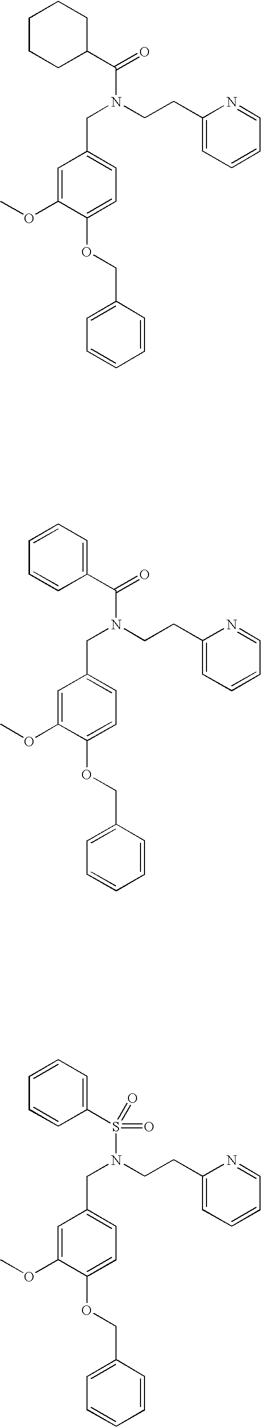

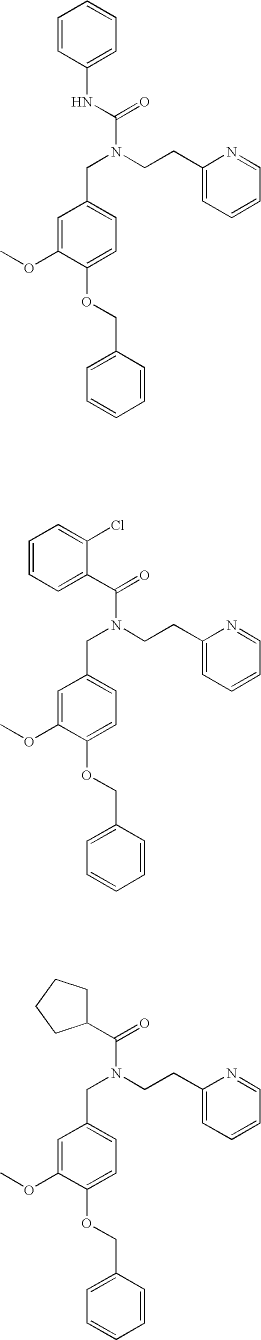

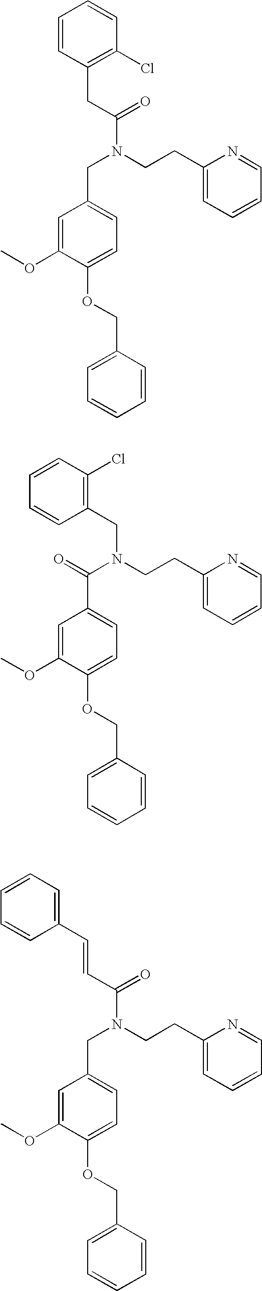

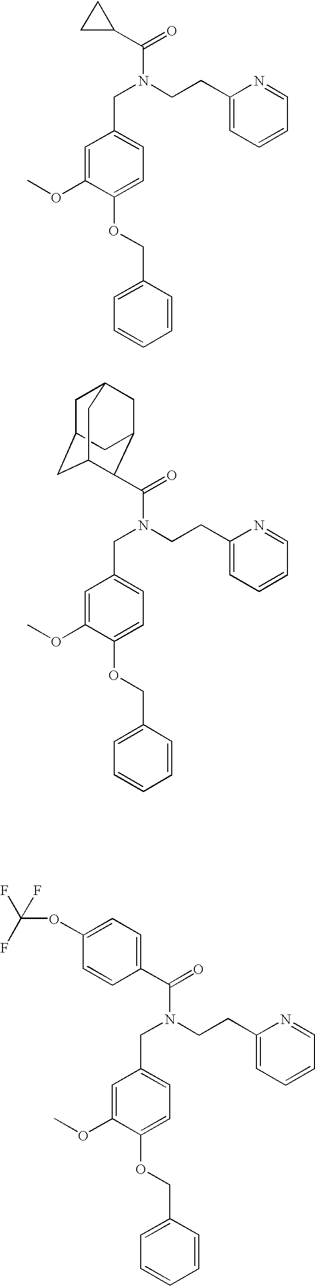

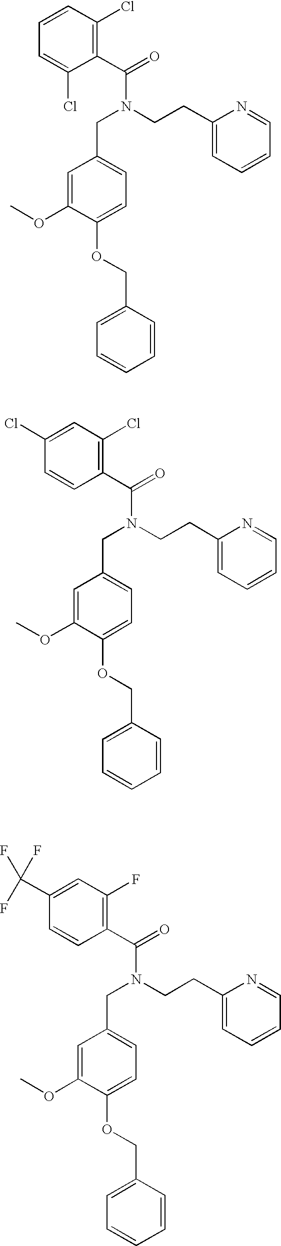

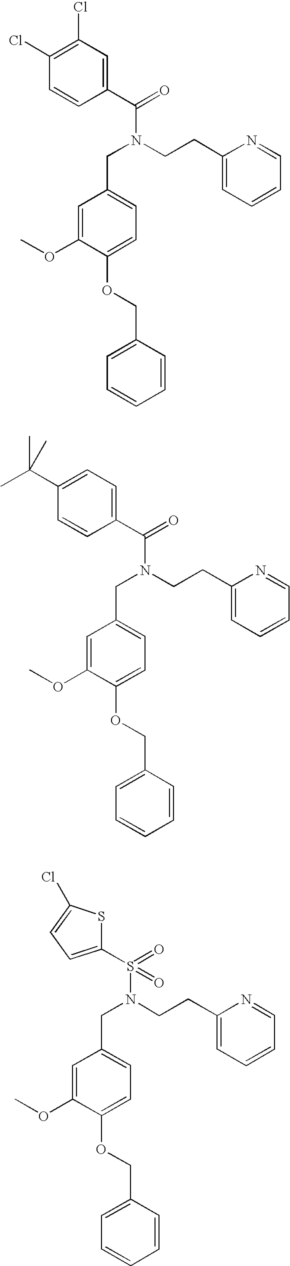

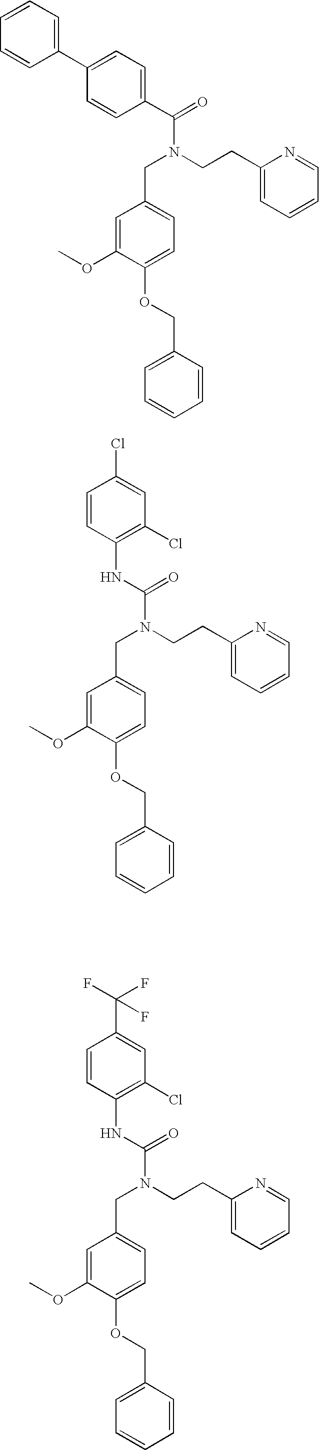

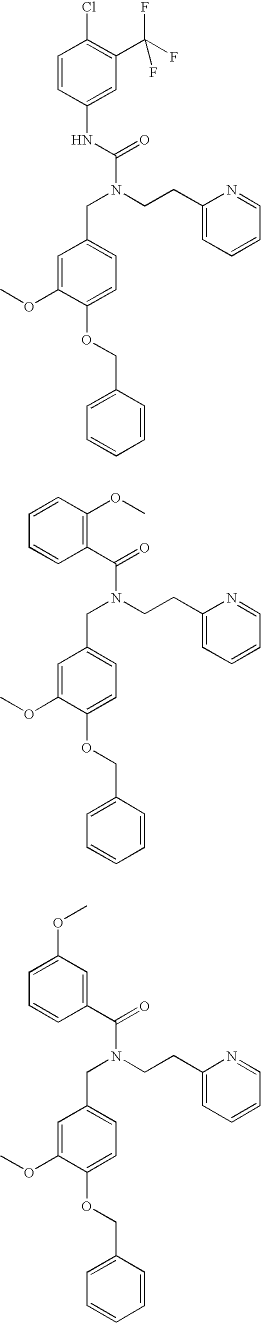

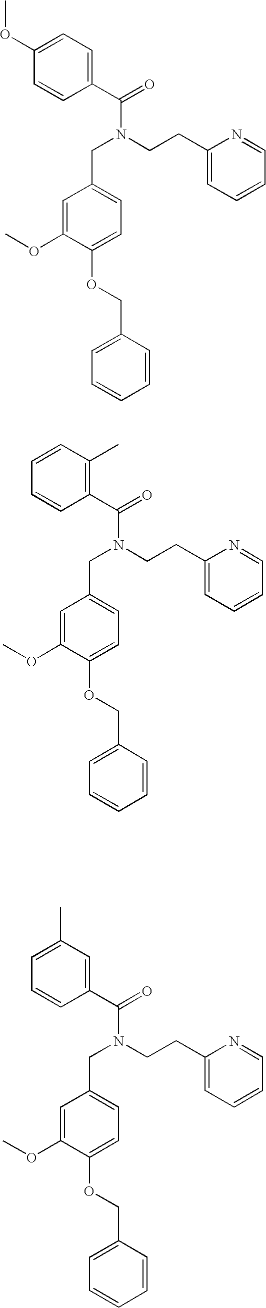

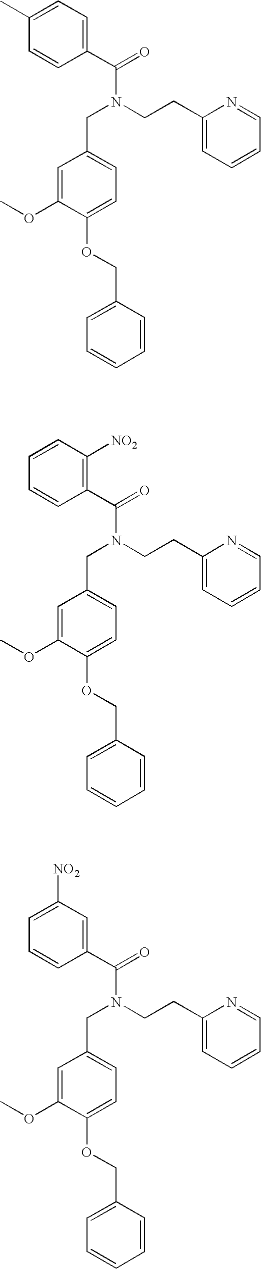

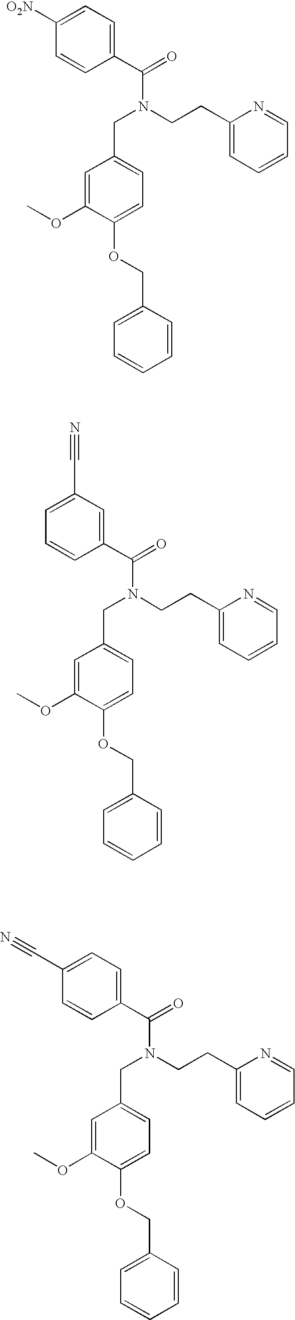

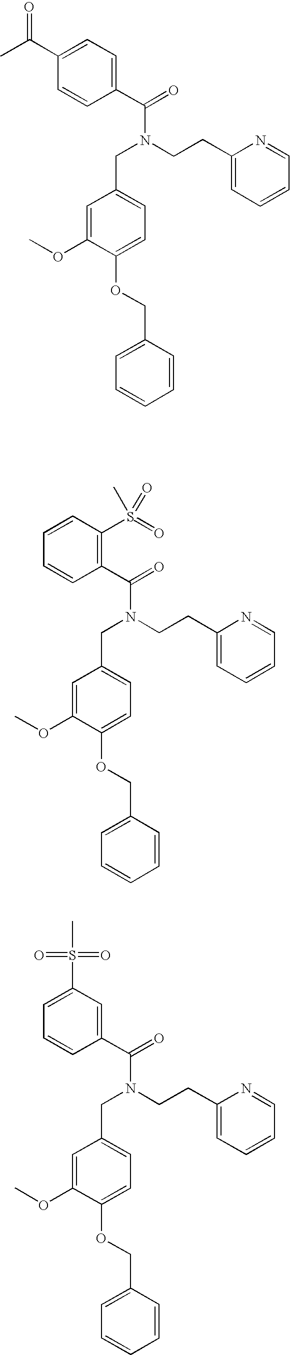

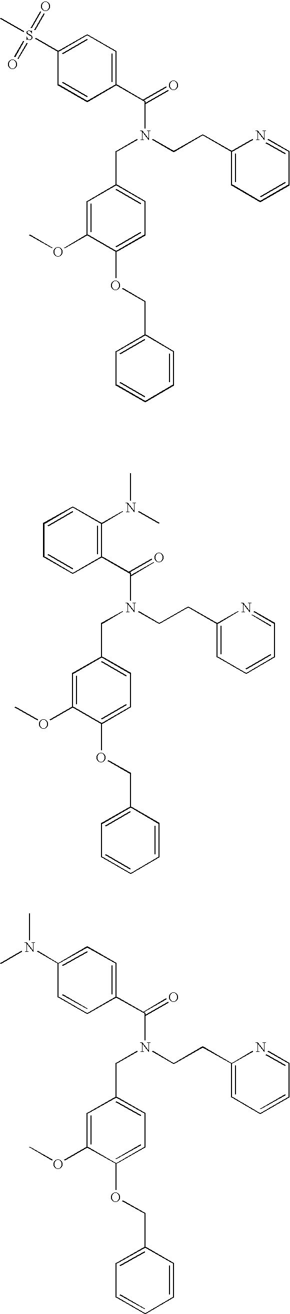

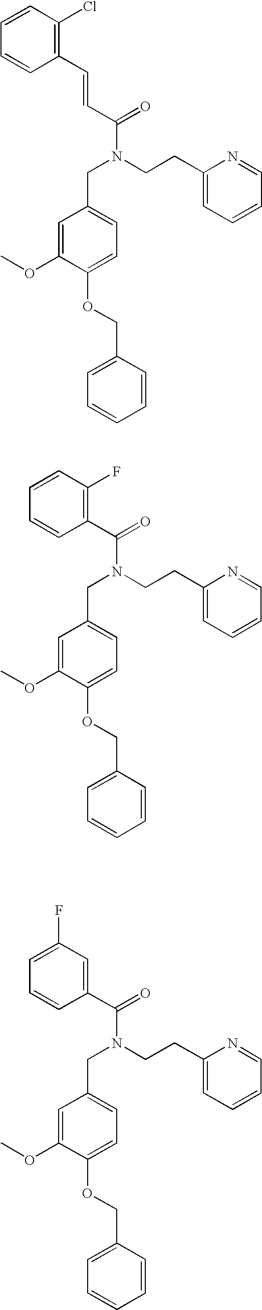

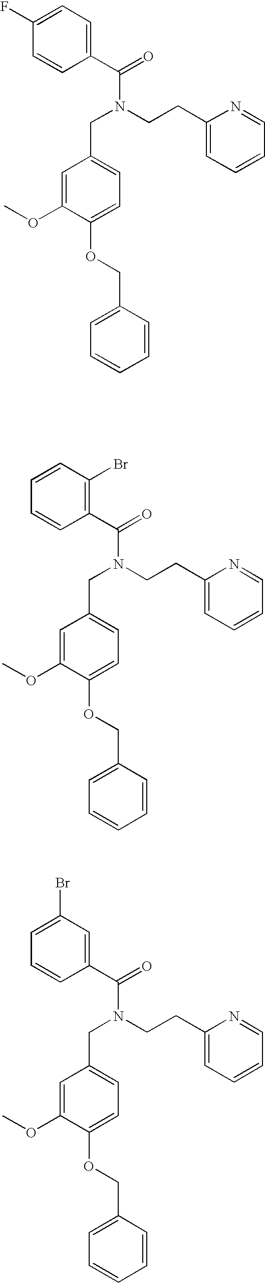

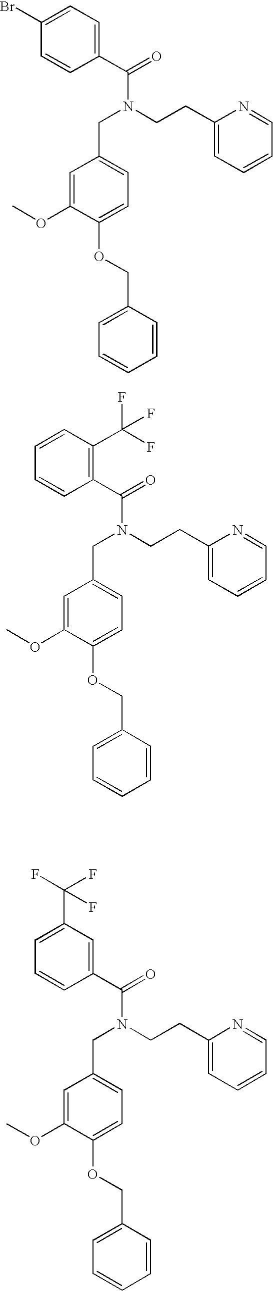

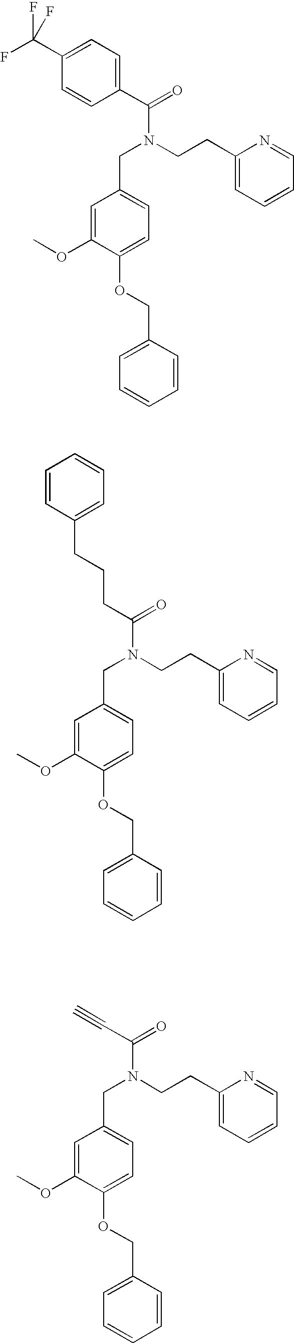

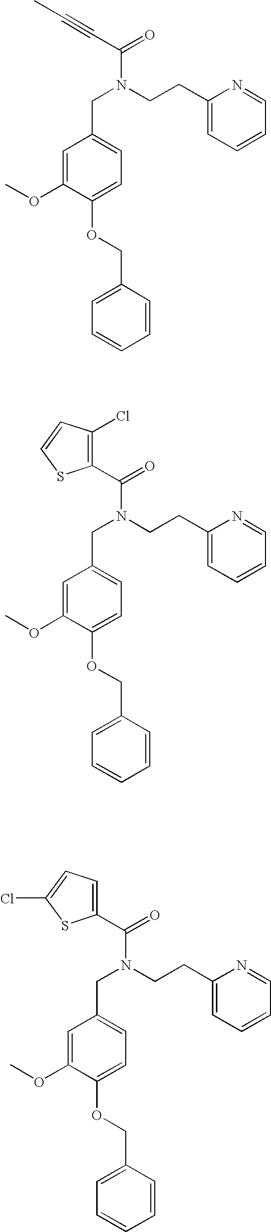

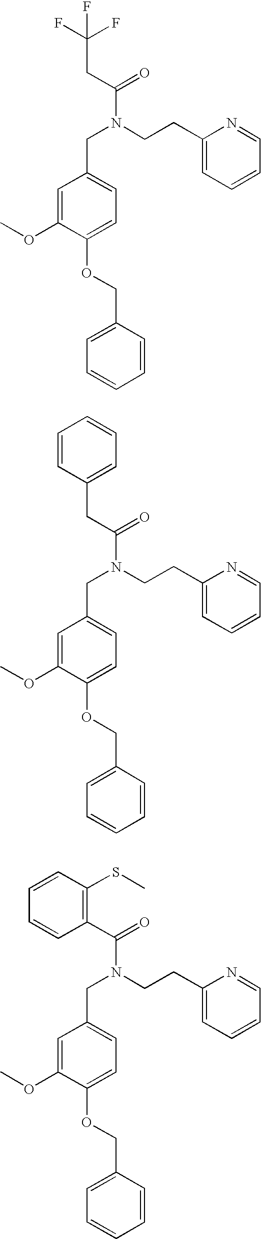

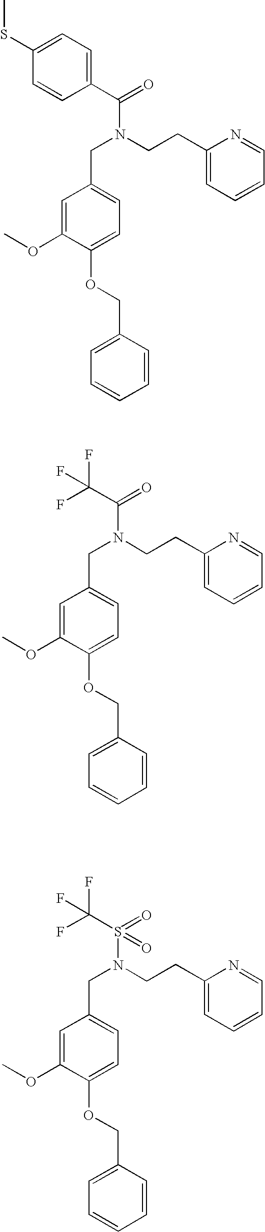

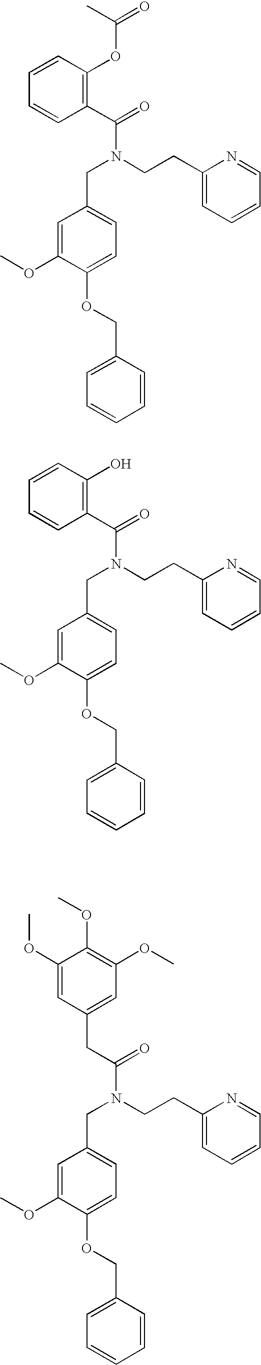

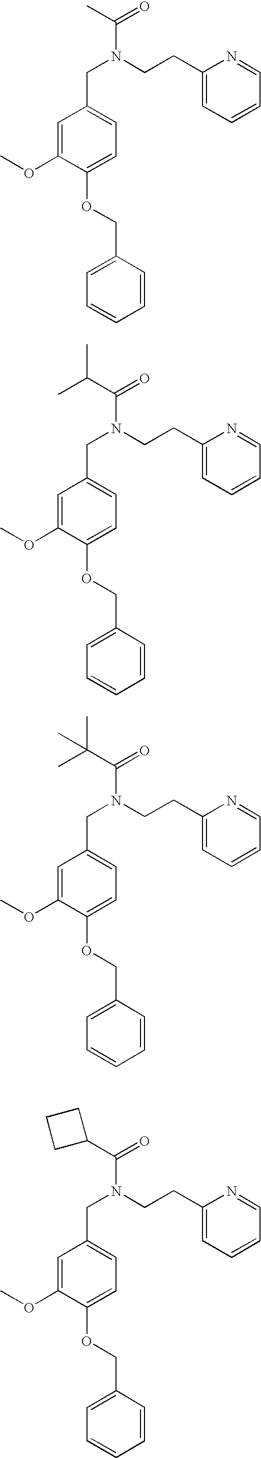

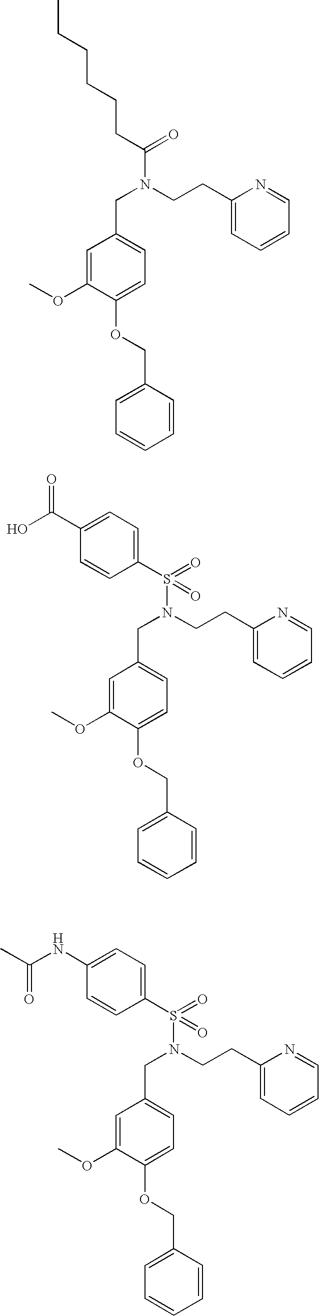

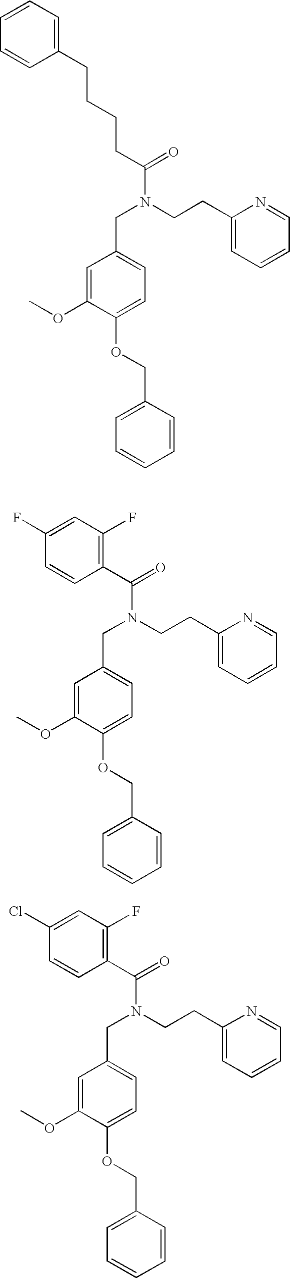

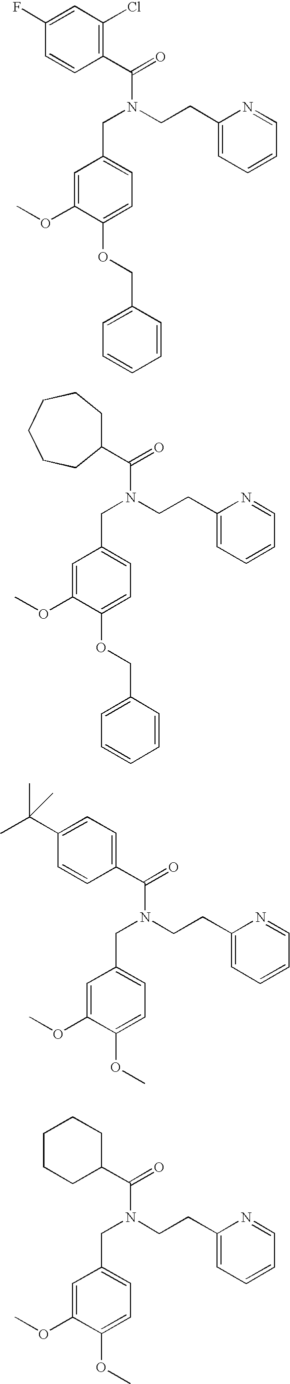

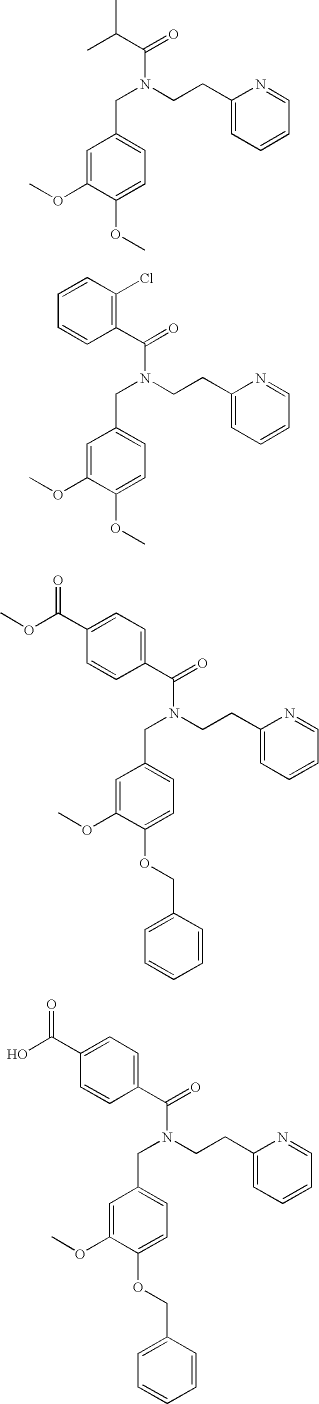

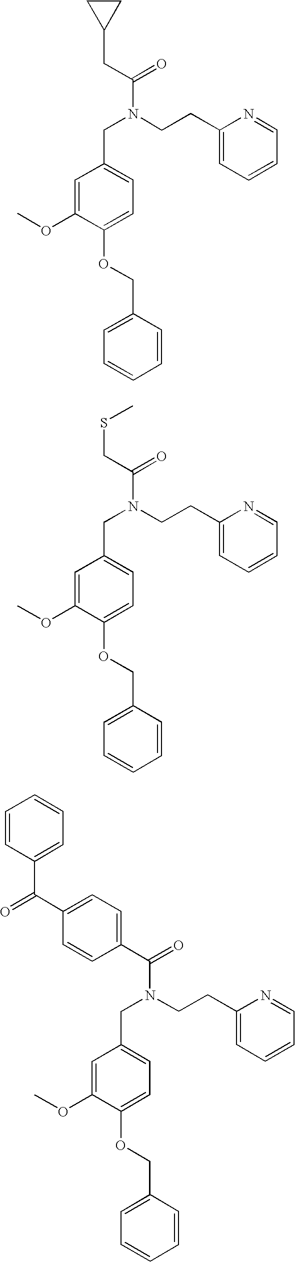

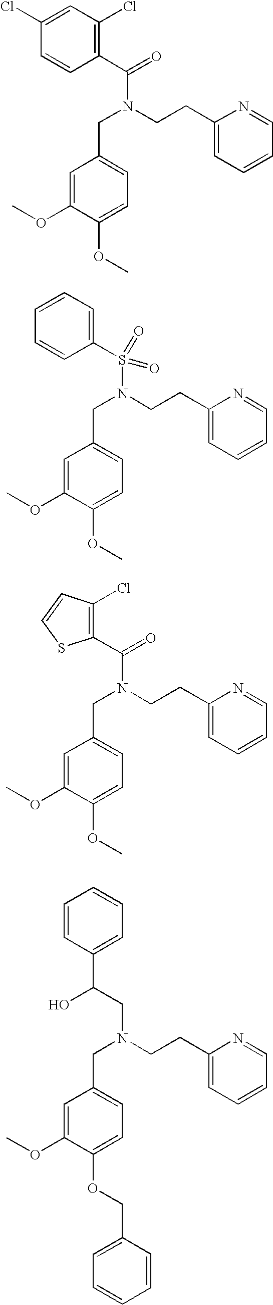

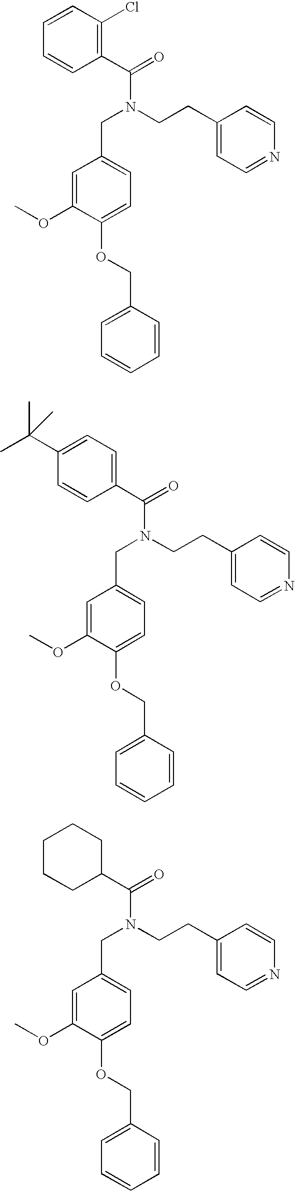

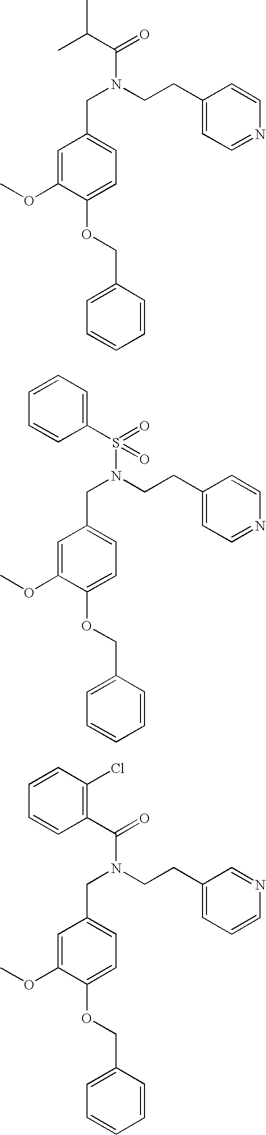

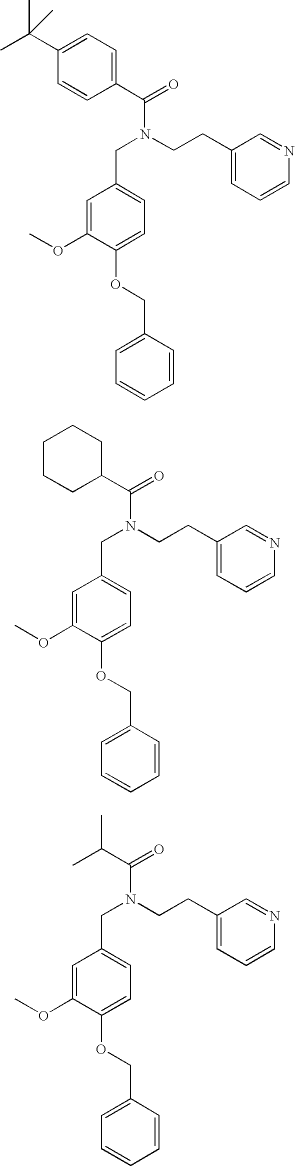

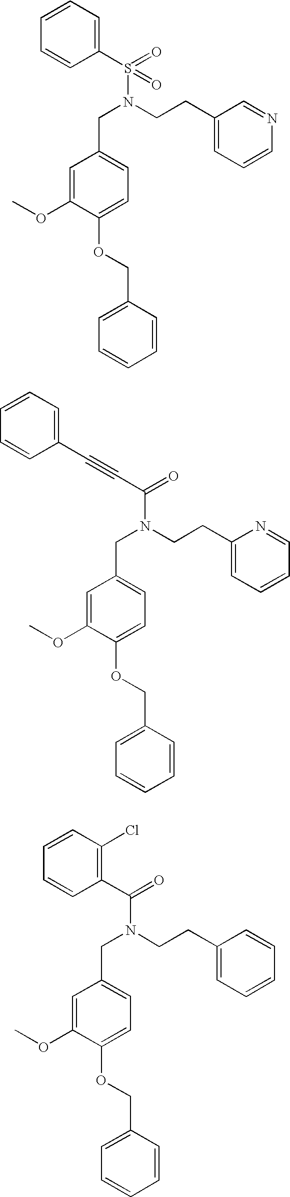

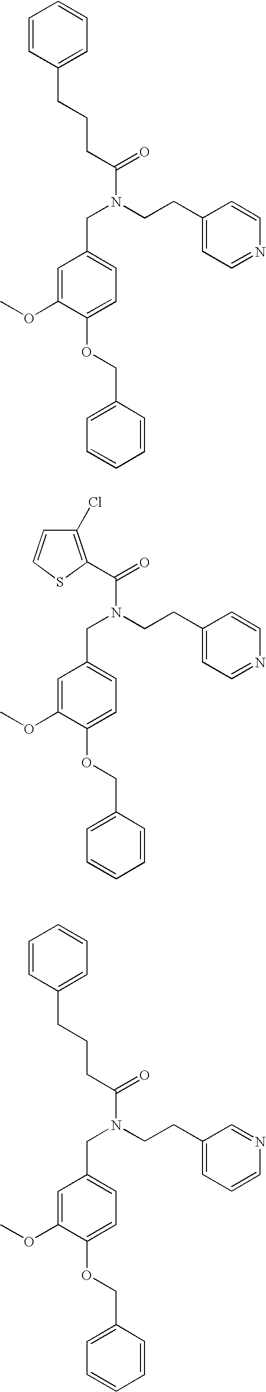

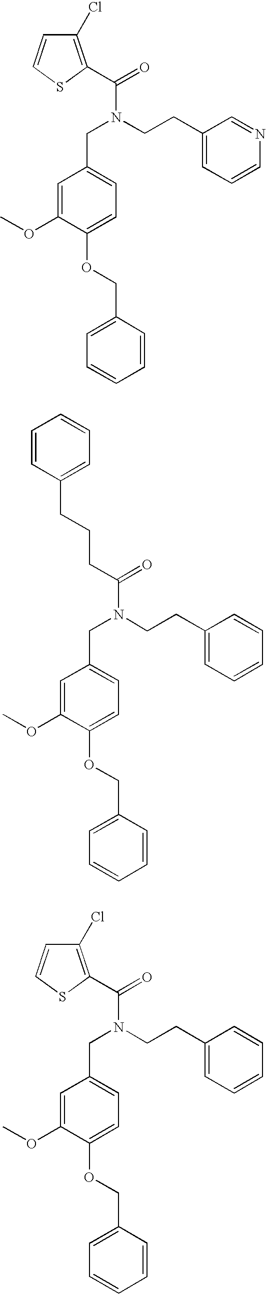

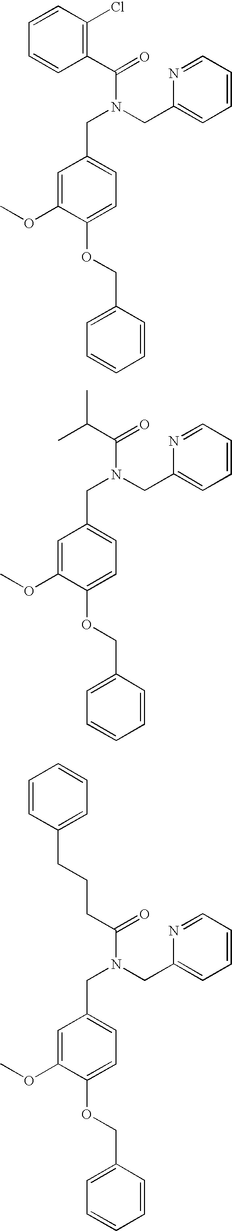

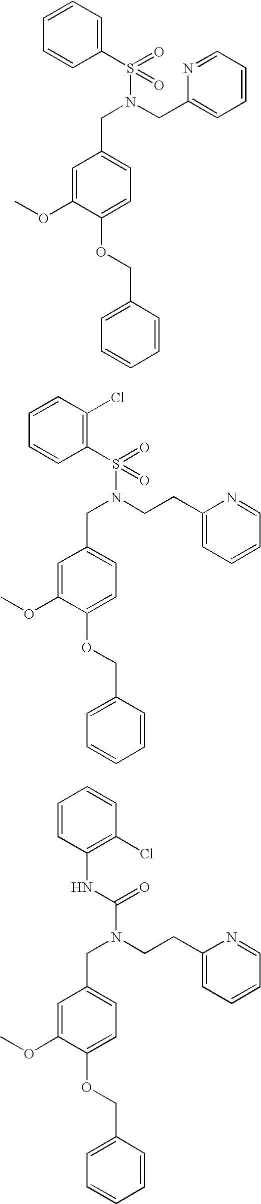

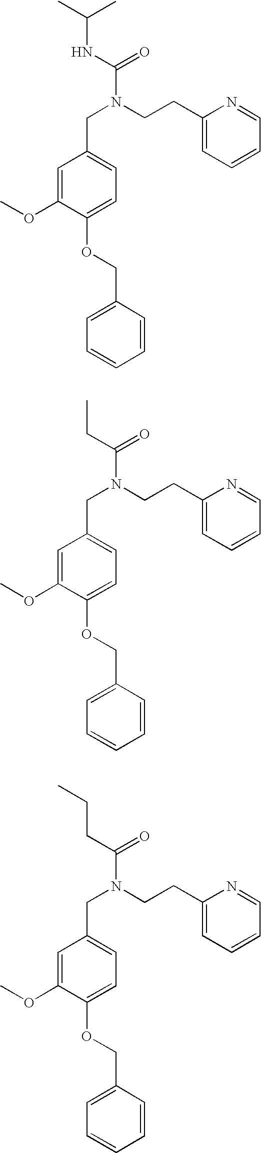

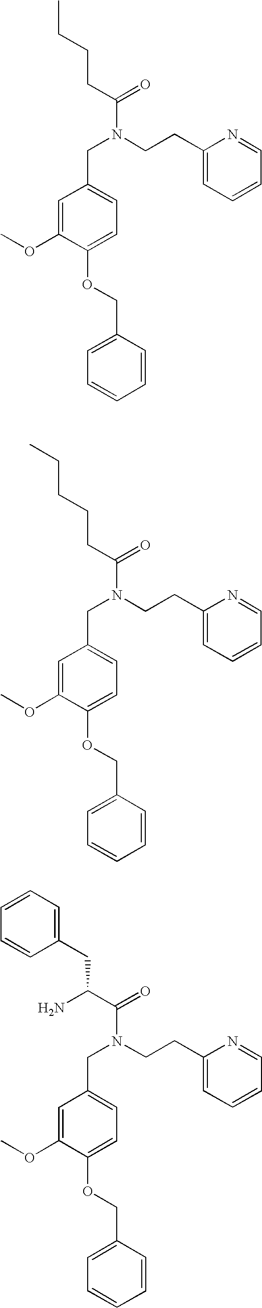

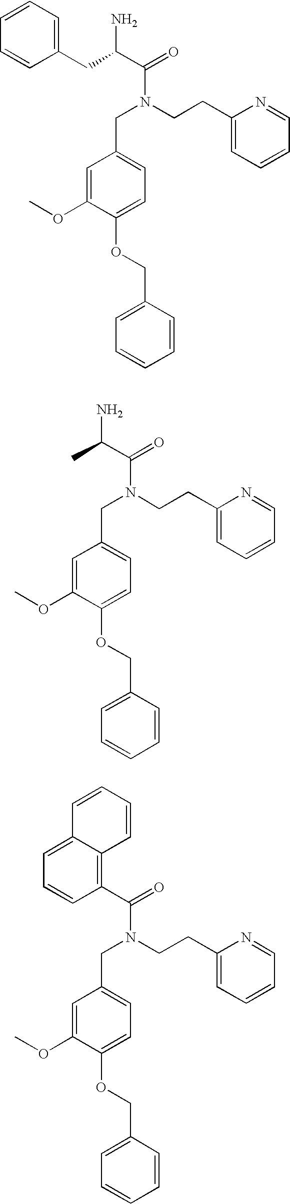

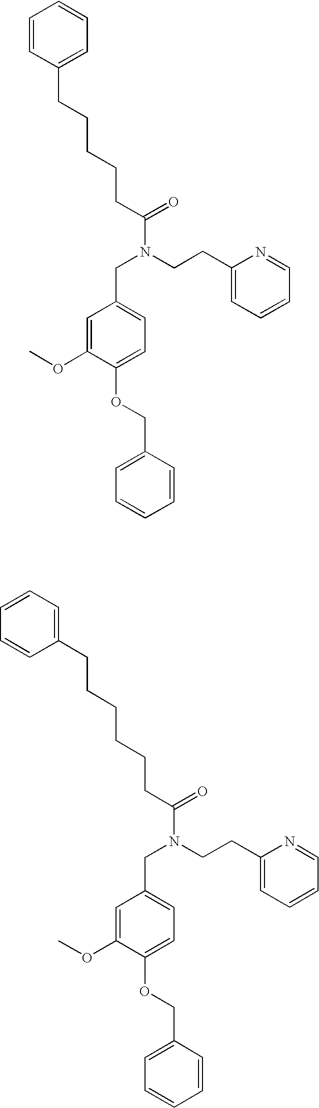

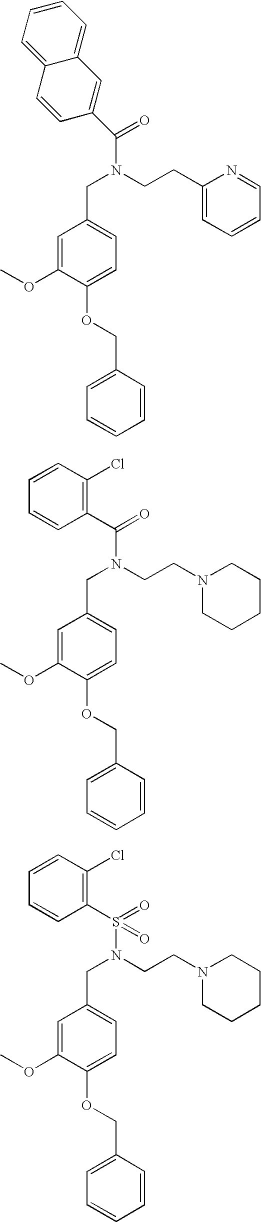

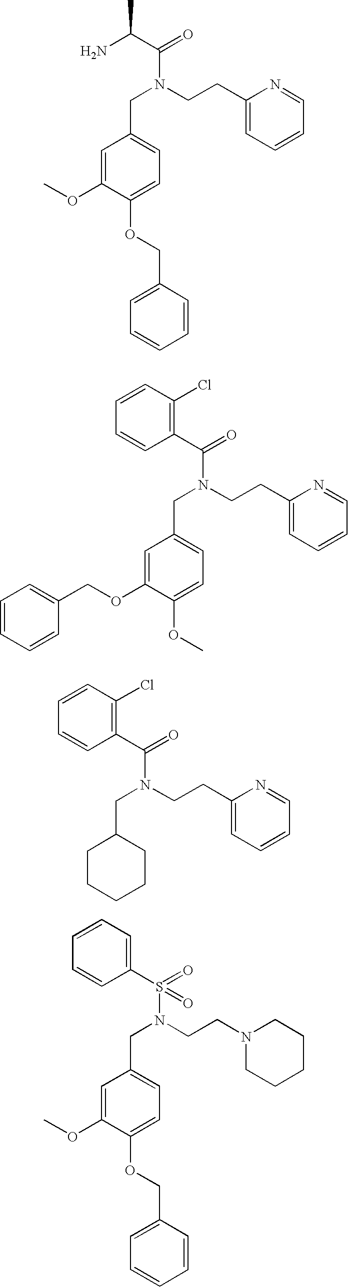

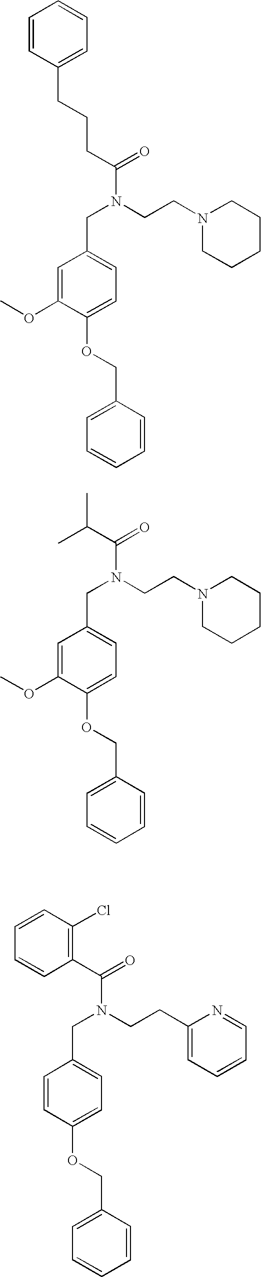

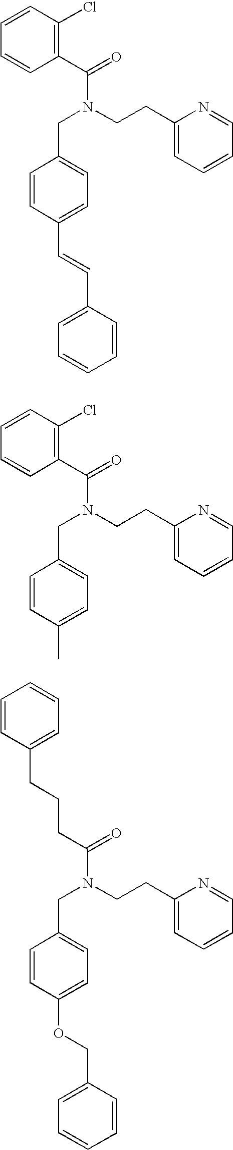

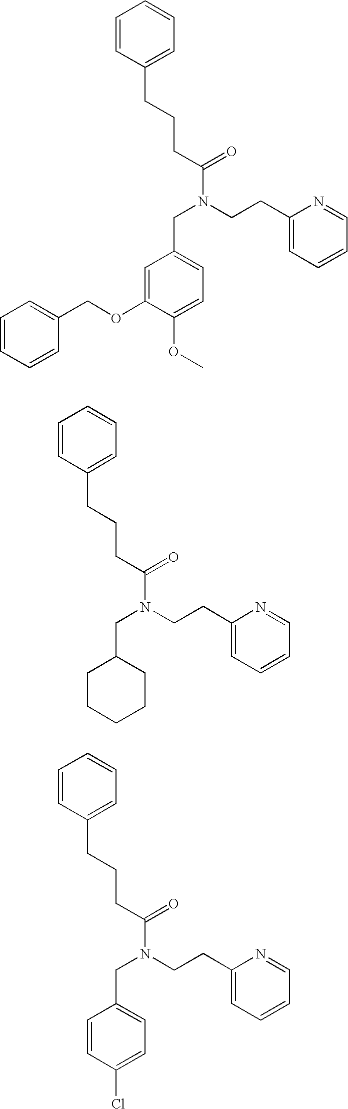

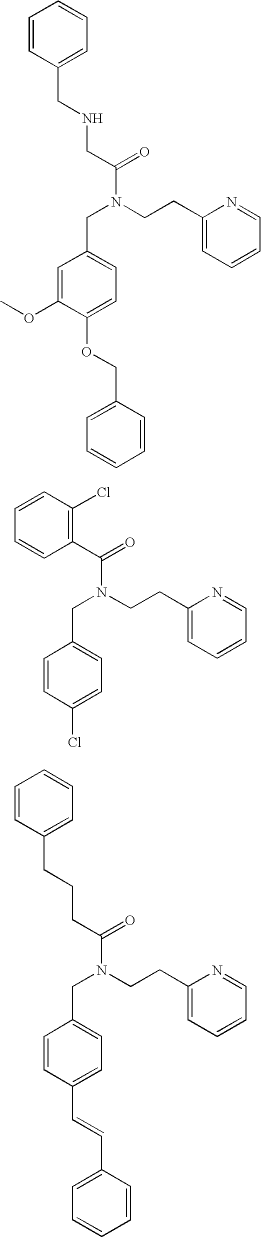

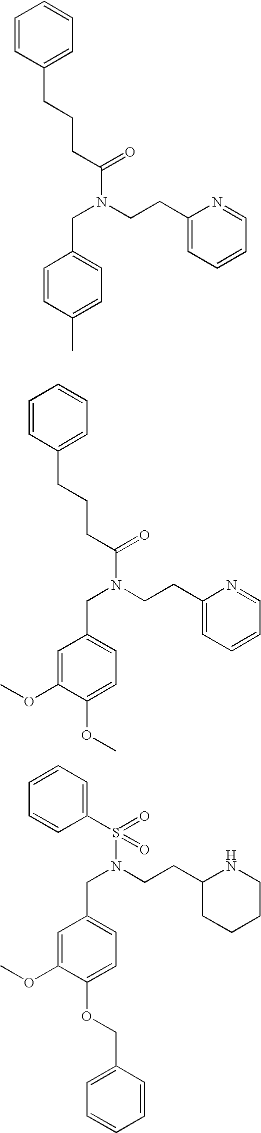

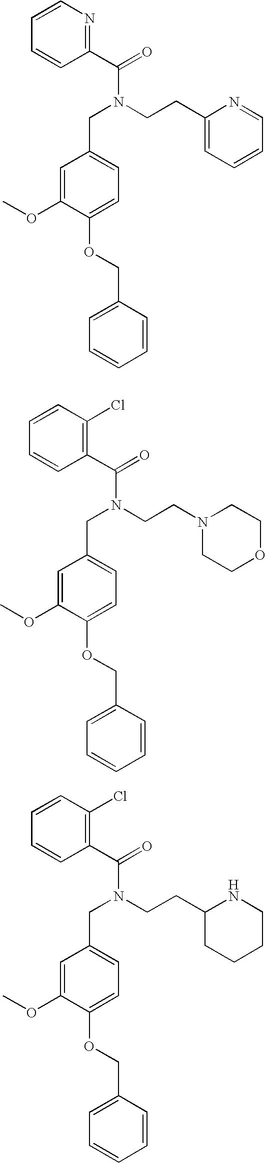

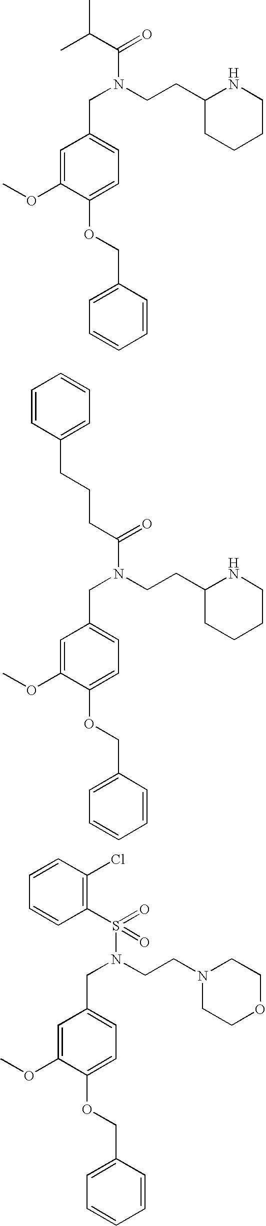

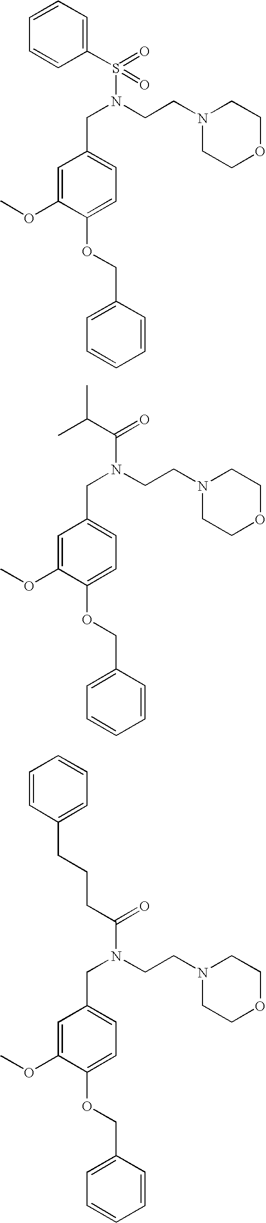

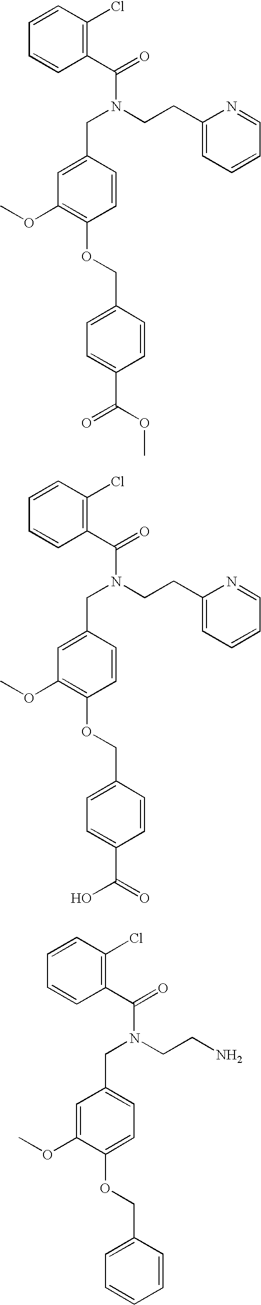

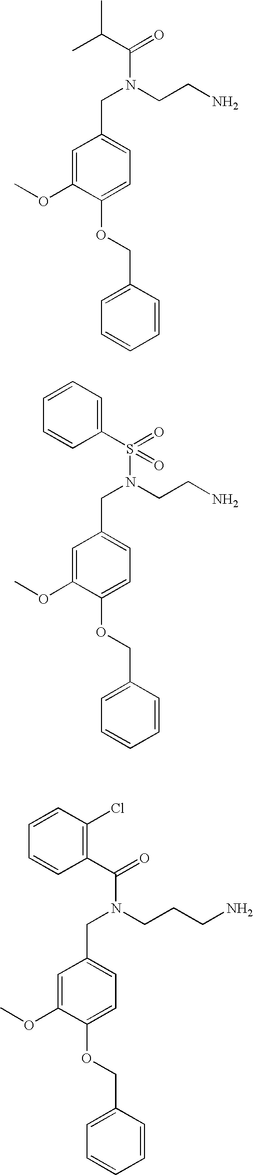

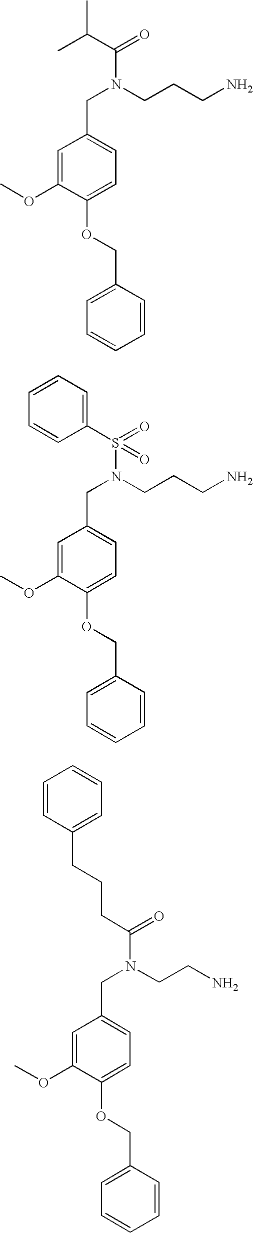

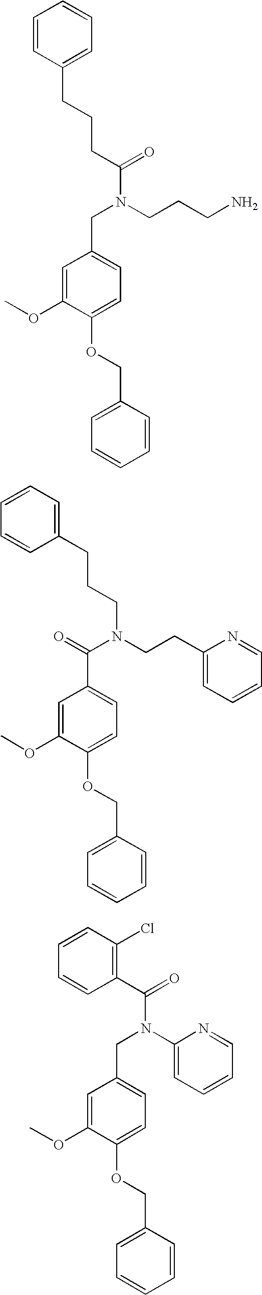

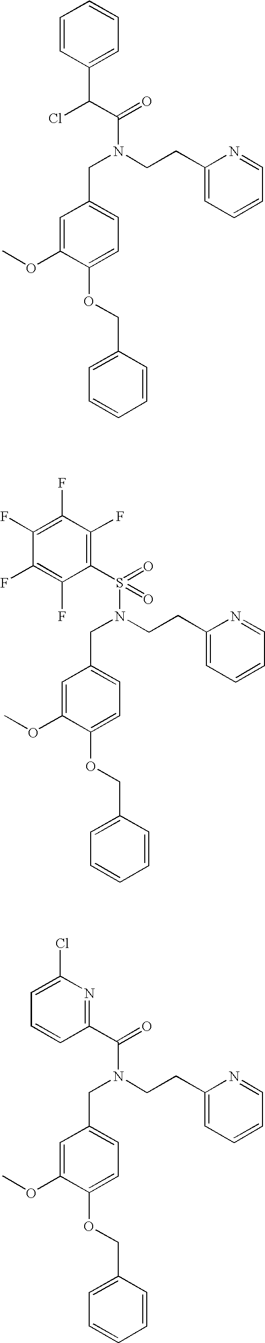

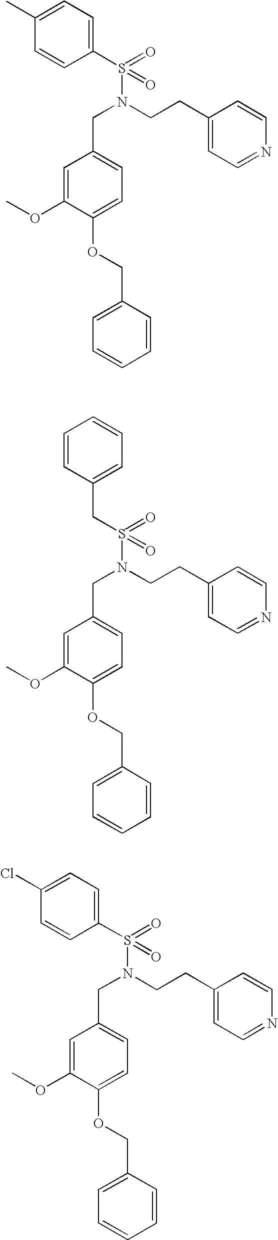

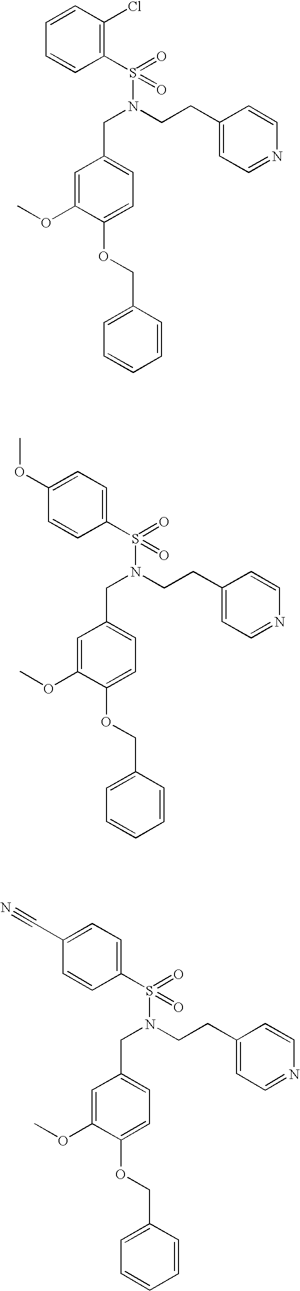

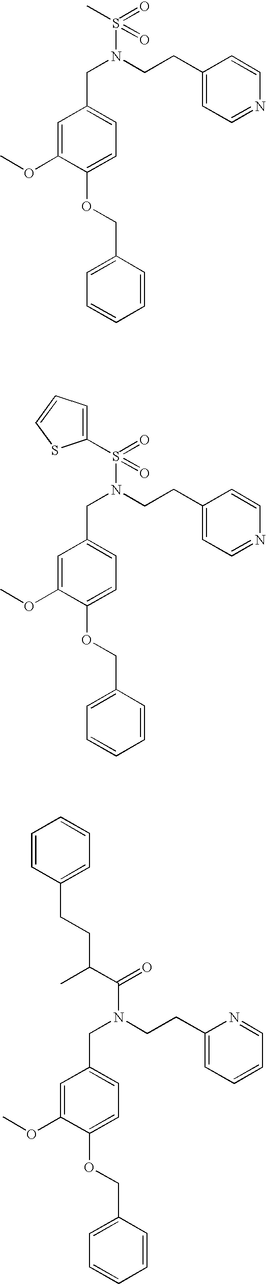

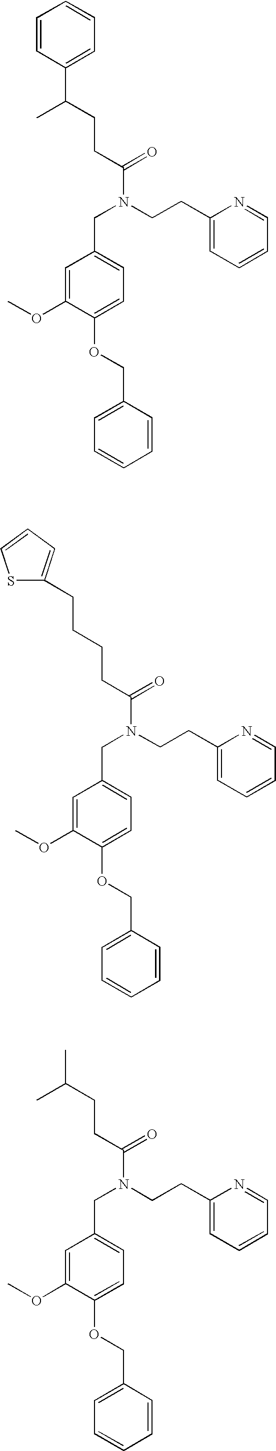

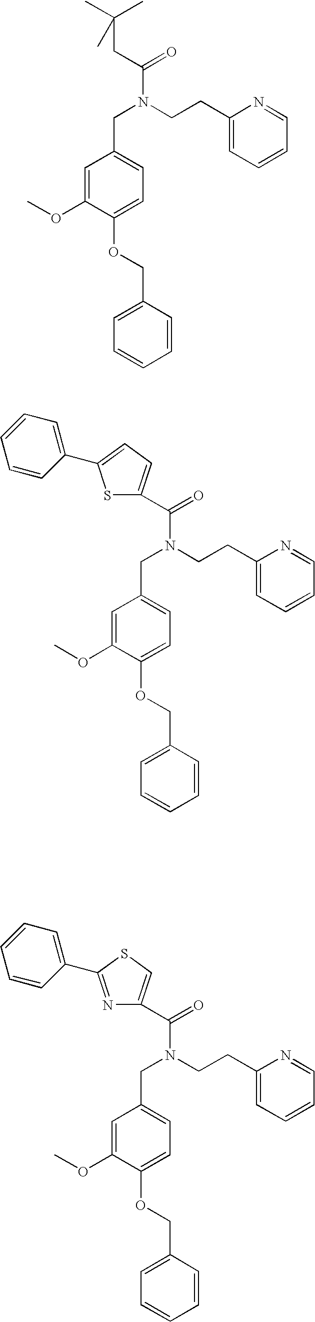

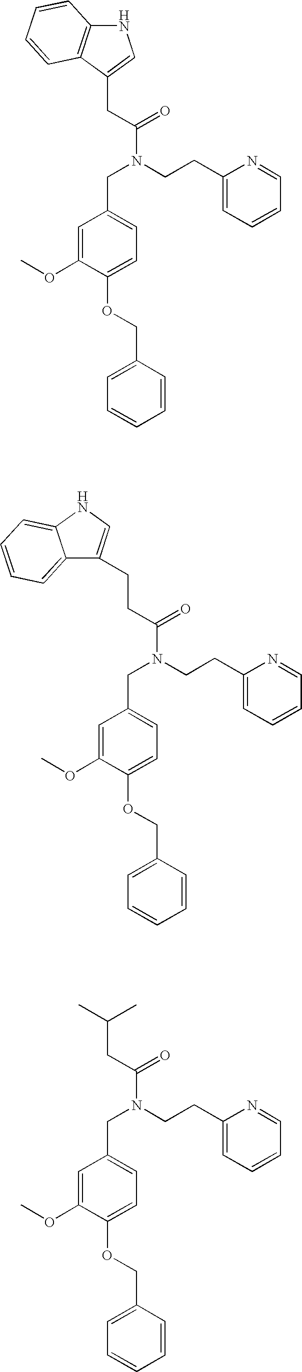

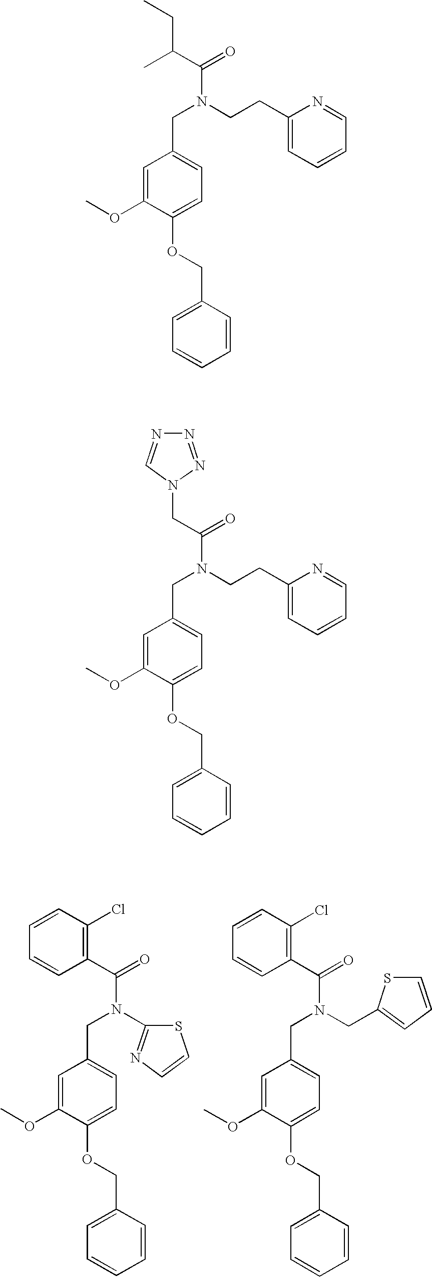

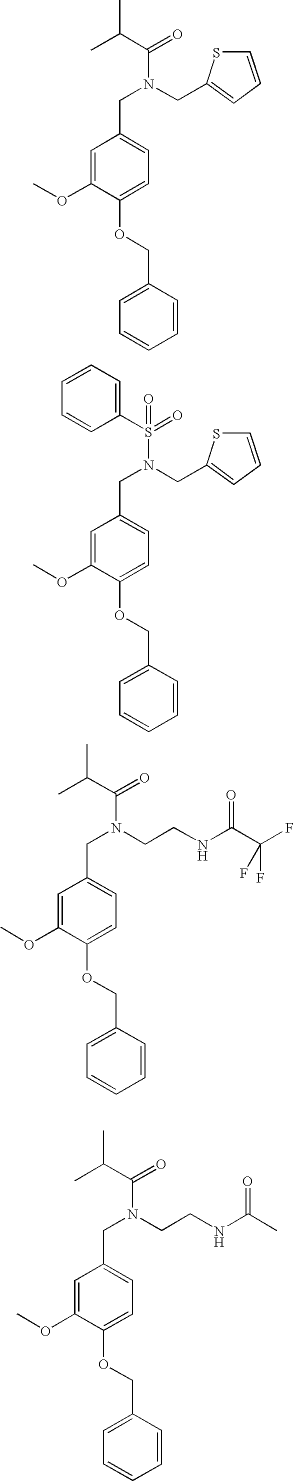

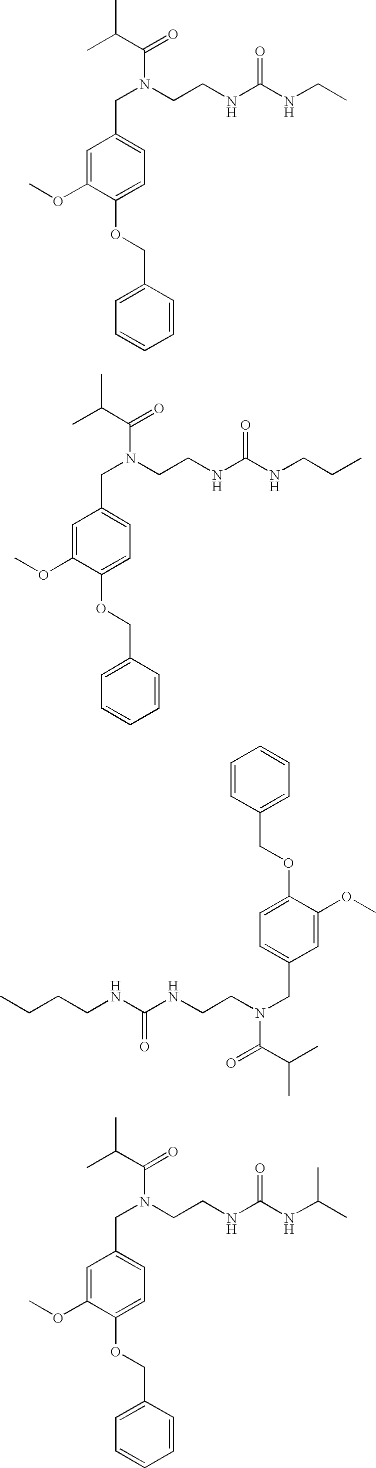

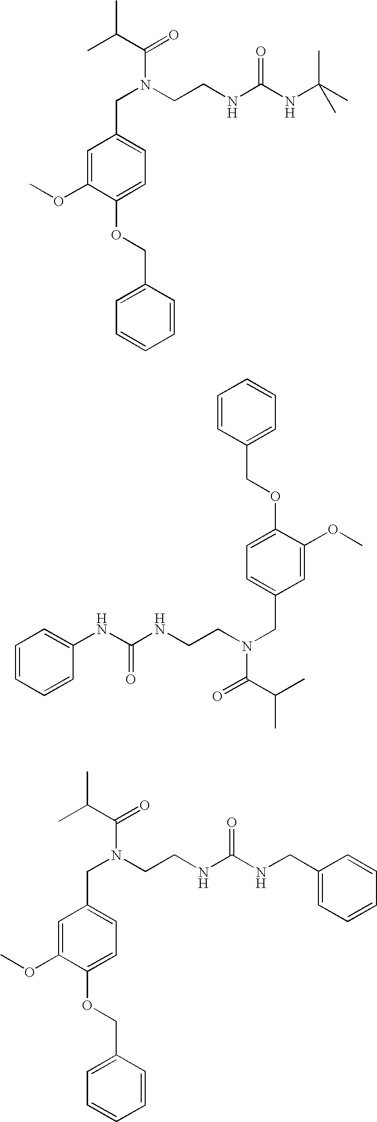

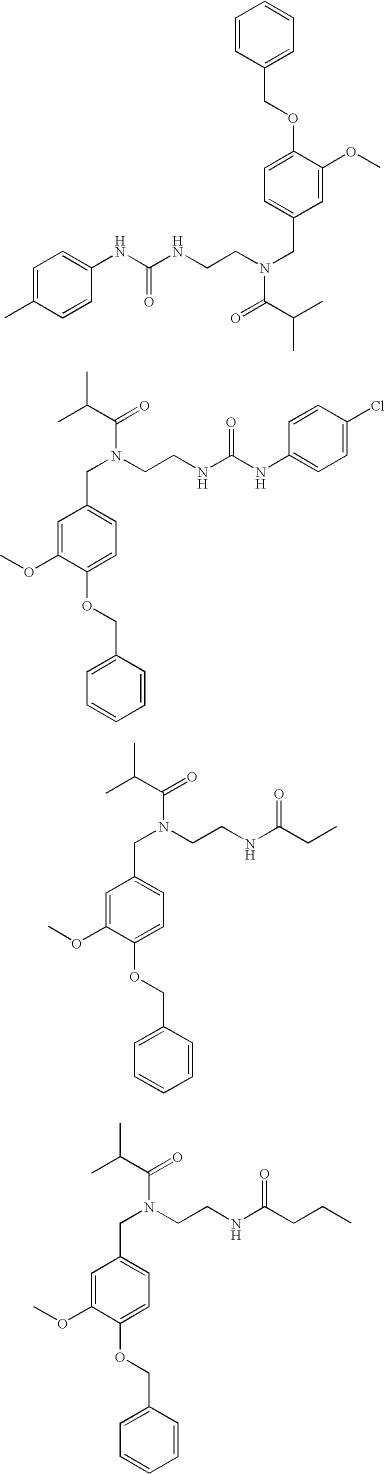

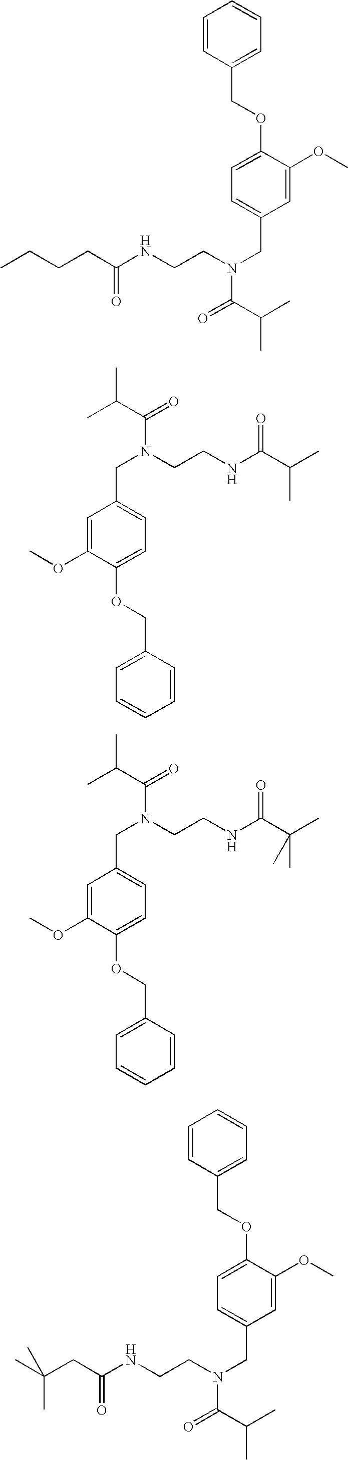

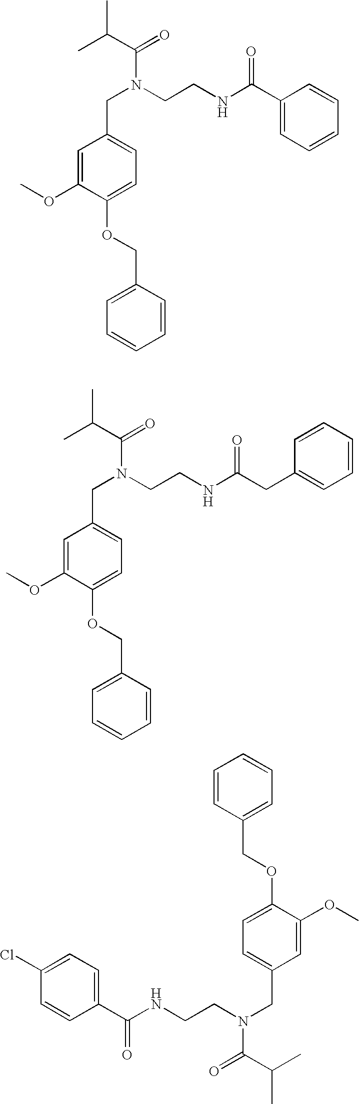

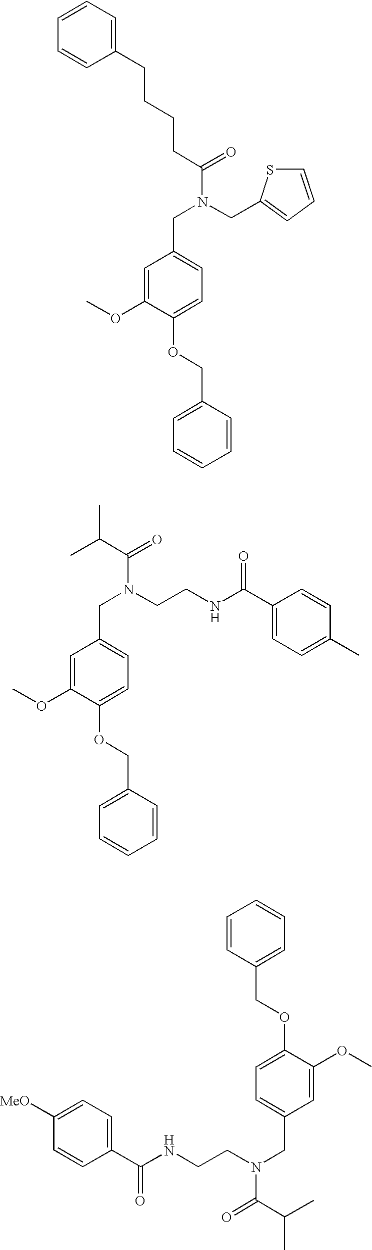

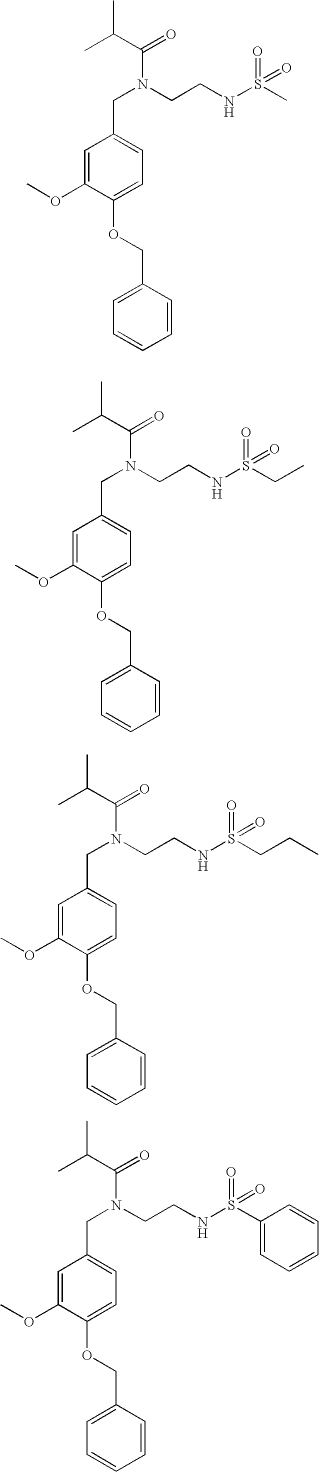

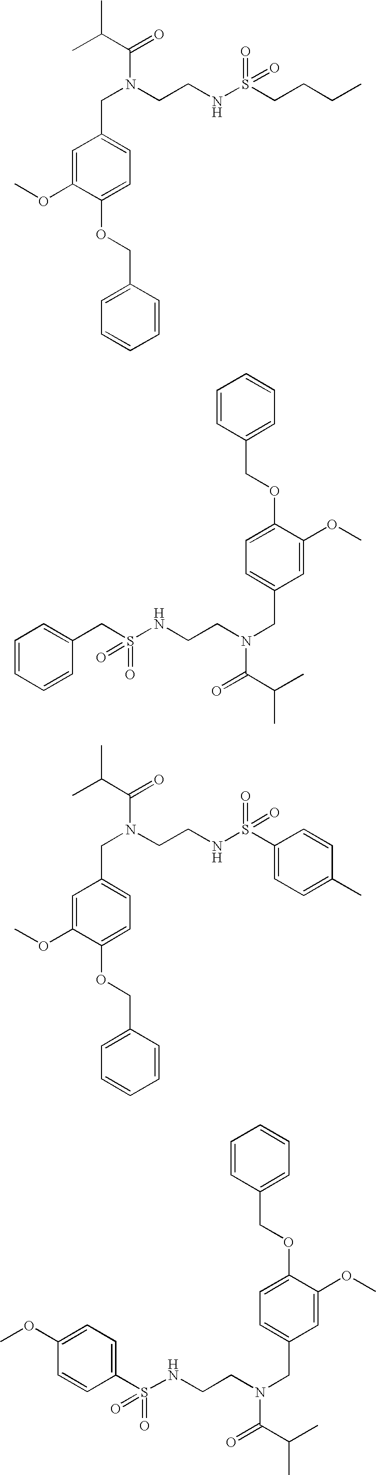

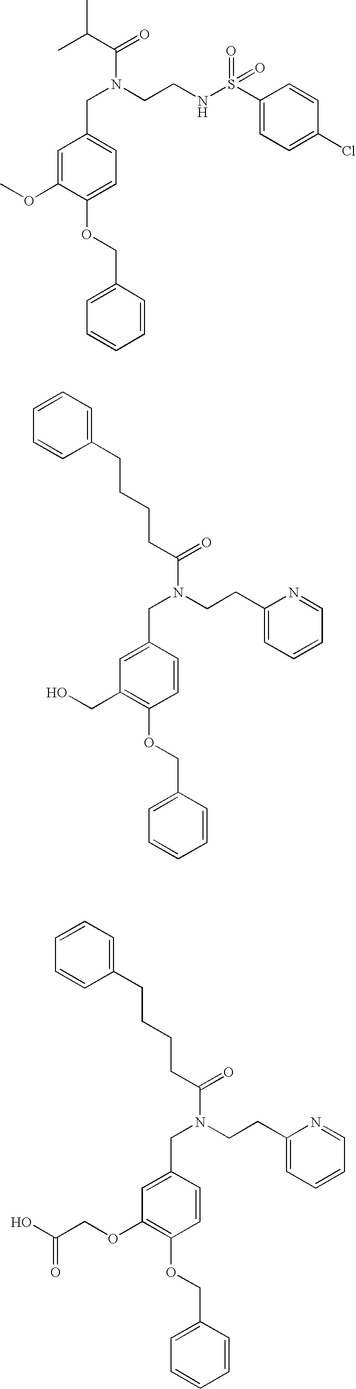

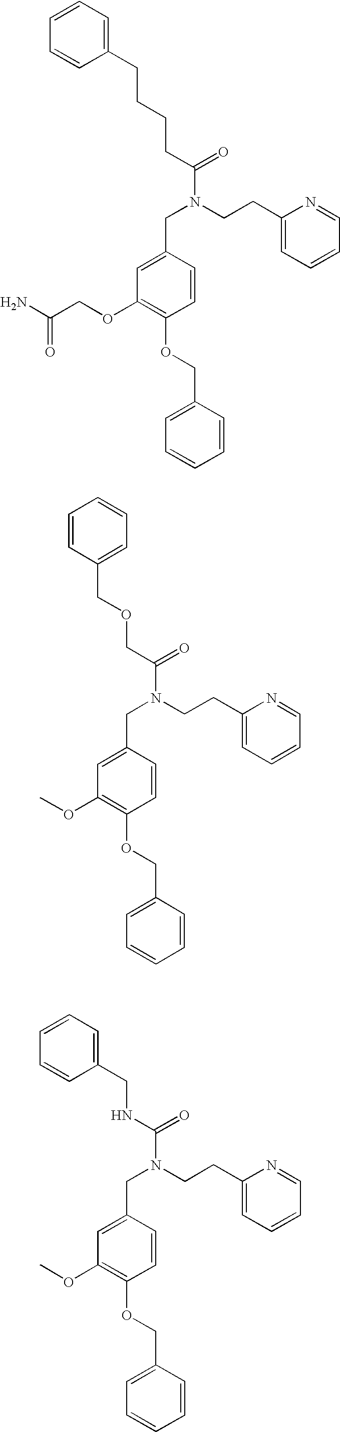

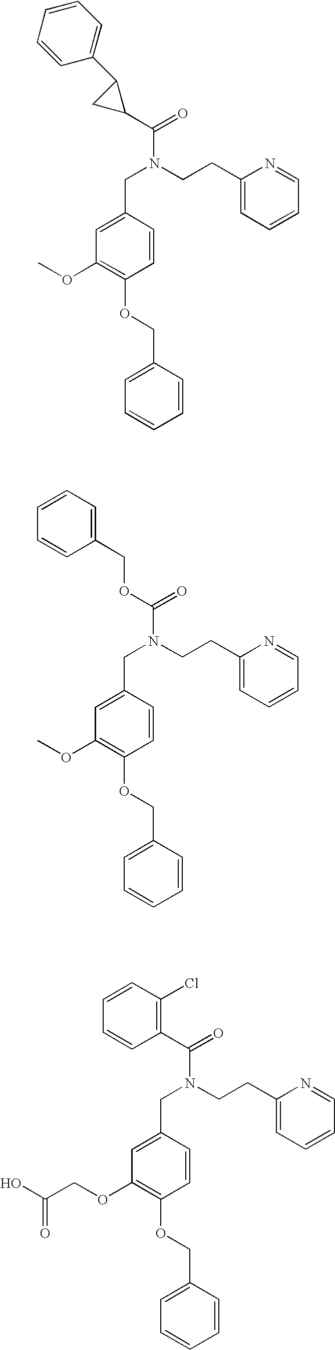

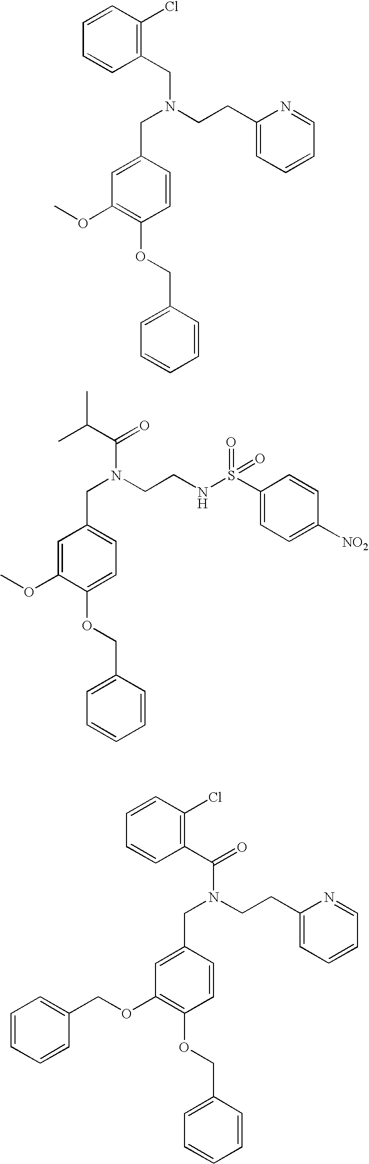

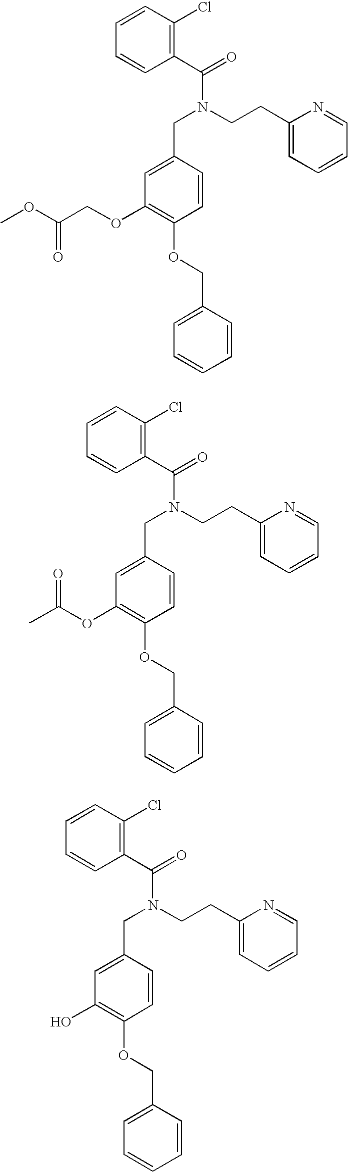

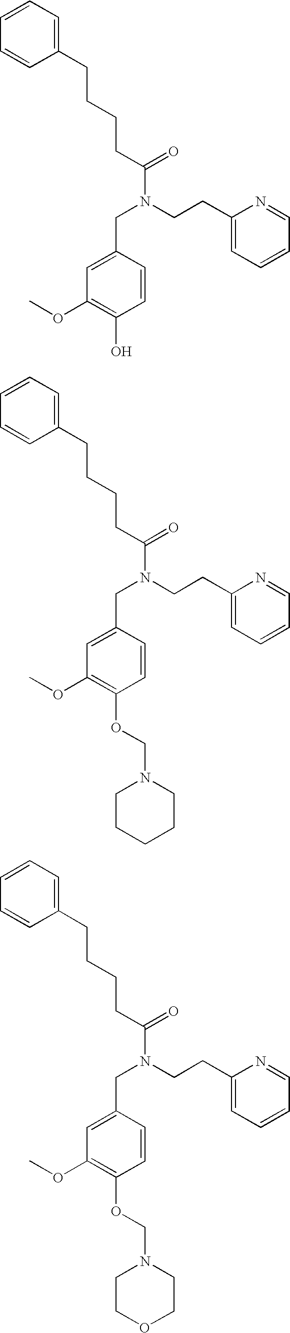

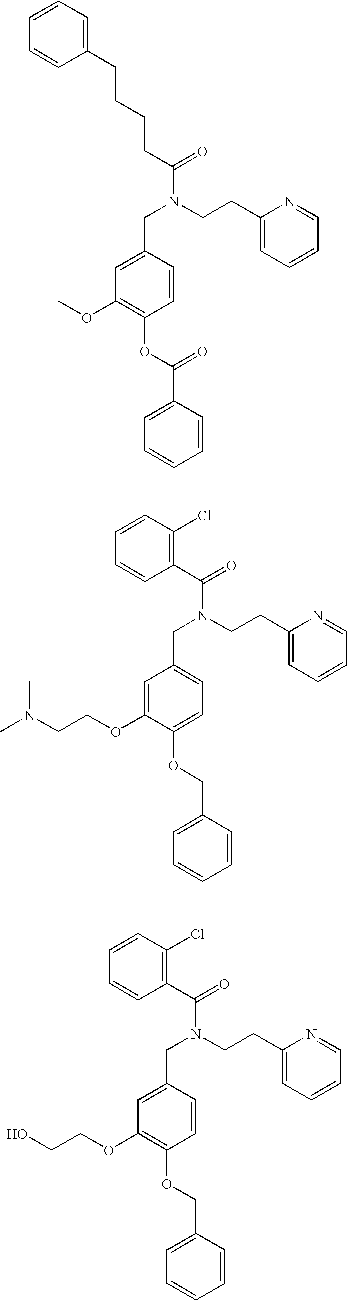

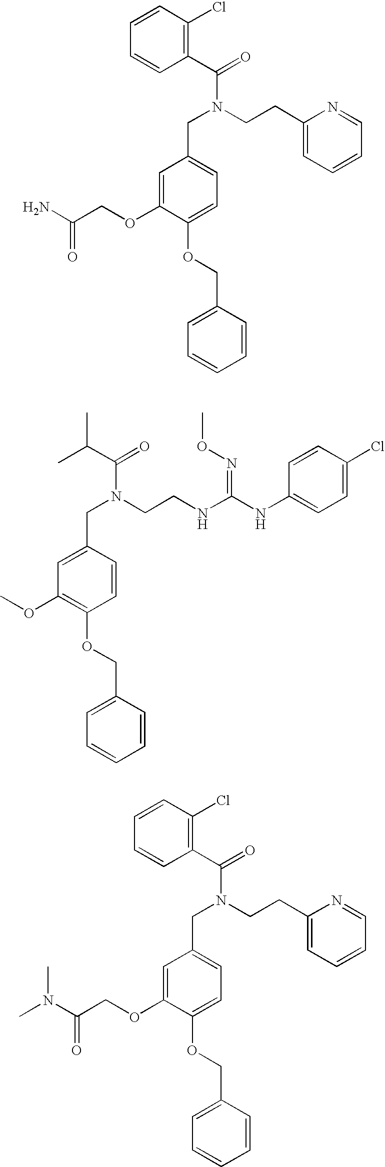

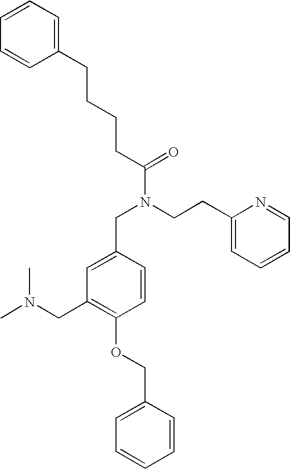

1. A compound comprising Formula (I) 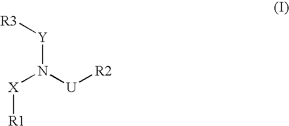
wherein
U is (CH₂)ₙ, CO, SO₂ or CONH;
n is 0, 1, 2, 3, 4 or 5;
X is CH₂, CO, SO₂ or CONH;
Y is CH₂, CO, SO₂ or CONH;
R1 is an optionally substituted aryl, aralkyl, heteroaryl or heteroarylalkyl;
R2 is an optionally substituted heteroalkyl, aryl, aralkyl, heteroaryl, heteroaralkyl, cycloalkyl, heterocycloalkyl or heteroalkylcycloalkyl and
R3 is an optionally substituted alkyl, alkenyl, alkinyl, heteroalkyl, cycloalkyl, alkylcycloalkyl, heterocycloalkyl, heteroalkylcycloalkyl, aryl, heteroaryl, heteroarylalkyl or aralkyl;
or a pharmacologically acceptable salt, solvate, hydrate or formulation thereof.

2. Compounds according to claim 1 wherein U is (CH₂)ₙ and n is 0, 1 or 2.

3. Compounds according to claim 1, wherein R1 is an optionally substituted phenyl ring.

4. Compounds according to claim 1, wherein R2 is a heterocycloalkyl or a heteroaryl group.

5. Compounds according to claim 1, wherein R2 is a pyridyl or a piperidyl group.

6. Compounds according to claim 1, wherein R3 is aryl or aralkyl.

7. Compounds according to claim 1, wherein R3 is a group of the Formula (CH₂)ₘPh wherein m is 0, 1, 2, 3, 4 or 5 and wherein the phenyl group may be optionally substituted.

8. Compounds according to claim 1, wherein X is CO or SO₂ and Y is CH₂.

9. Compounds according to claim 1, wherein X is CH₂ and Y is CO or SO₂.

10. A pro-drug comprising a compound according to claim 1, and at least one pharmacologically acceptable protective group, wherein the protecting group is cleavable under physiological conditions.

11. A pharmaceutical composition comprising a compound, a pharmacologically acceptable salt, a solvates, a hydrate or a prodrug according to claim 1 and optional carriers, adjuvants or diluents.

12. A method for the treatment of cancer comprising to a subject in need administering thereof an effective amount of a compound of claim 1 or a prodrug of claim 10.
